(19) United States
(12) Patent Application Publication
(10) Pub. No.: US 2024/0325319 A1
(43) Pub. Date: Oct. 3, 2024

(54) INHIBITORS OF AMYLOID BETA OLIGOMERIZATION AND THERAPEUTIC USES THEREOF

(71) Applicant: Northwestern University, Evanston, IL (US)

(72) Inventors: Richard B. Silverman, Winnetka, IL (US); William L. Klein, Evanston, IL (US); Elizabeth A. Johnson, Evanston, IL (US)

(21) Appl. No.: 18/579,307

(22) PCT Filed: Jul. 14, 2022
(86) PCT No.: PCT/US22/73747

§ 371 Date: Jan. 12, 2024

Related U.S. Application Data
(60) Provisional application No. 63/203,245, filed on Jul. 14, 2021.

(51) Int. Cl. A61K 31/122; A61K 9/00; A61K 31/13; A61K 31/27; A61K 31/445; A61K 31/55; A61P 25/28; C12N 5/0793; G01N 33/50; G01N 33/68

(52) U.S. Cl. CPC A61K 31/122; A61K 9/0053; A61K 31/13; A61K 31/27; A61K 31/445; A61K 31/55; A61P 25/28; C12N 5/0619; G01N 33/5058; G01N 33/6896; C12N 2501/998; G01N 2333/4709

(57) ABSTRACT

Disclosed are compounds, pharmaceutical compositions comprising the compounds, and methods of using the compounds and pharmaceutical compositions for treating and/or preventing a disease or disorder associated with amyloid beta biological activity in a subject in need thereof. The disclosed compounds include cyclohexane 1,3-diones which may inhibit one or more biological activities of amyloid beta, such as amyloid beta oligomerization. As such, the disclosed compounds and pharmaceutical compositions may be utilized in methods for treating and/or preventing a disease or disorder that is associated with amyloid beta activity in a subject in need thereof, which diseases and disorders may be associated with amyloid beta oligomerization.

### CROSS-REFERENCE TO RELATED APPLICATIONS

This application claims benefit of priority to U.S. Patent Application Ser. No. 63/203,245, filed Jul. 14, 2021, the contents of which is incorporated by reference in its entirety.

### REFERENCE TO AN ELECTRONIC SEQUENCE LISTING

The contents of the electronic sequence listing (702581.02174.xml; Size: 5,531 bytes; and Date of Creation: Jul. 13, 2022) is herein incorporated by reference in its entirety.

### STATEMENT REGARDING FEDERALLY SPONSORED RESEARCH OR DEVELOPMENT

This invention was made with government support under AG061708 and AG050492 awarded by the National Institutes of Health. The government has certain rights in the invention.

### BACKGROUND

The field of the invention relates to small molecule inhibitors of amyloid beta (A3) oligomerization and the use thereof in treating diseases and disorders associated with A3 oligomerization.

Alzheimer's disease currently afflicts 5.8 million Americans. In the United States, it is the sixth leading cause of death. Oligomers of amyloid beta peptide and hyper-phosphorylated tau are heavily implicated in Alzheimer's memory loss. Amyloid beta oligomers are observed in the brains of Alzheimer's patients. These amyloid beta oligomers bind to neurons and induce tau phosphorylation and neuronal damage.

Currently, no FDA approved treatment for Alzheimer's disease can slow or stop the progression of disease, and none acts directly on toxic oligomers. Therefore, there is a need in the field for therapeutics that target the pathogenic amyloid beta oligomers.

### SUMMARY

Disclosed herein is the use of (S)-5-(1-(3,5-bis(trifluoromethyl)phenoxy)ethyl)cyclohexane-1,3-dione, or a pharmaceutically acceptable salt thereof, in methods for treatment, modulating amyloid-beta oligomerization, and detecting a candidate compound. Also disclosed is a pharmaceutical composition or unit dosage package comprising (S)-5-(1-(3,5-bis(trifluoromethyl)phenoxy)ethyl)cyclohexane-1,3-dione, or a pharmaceutically acceptable sale thereof. (S)-5-(1-(3,5-bis(trifluoromethyl)phenoxy)ethyl)cyclohexane-1,3-dione has a structural formula of

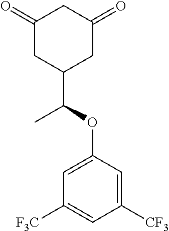 [SMILES: C[C@H](Oc1cc(C(F)(F)F)cc(C(F)(F)F)c1)C1CC(=O)CC(=O)C1]

and may be referred to as NU-9.

In one aspect of the current disclosure, methods of treating Alzheimer's disease in a subject in need thereof are provided. In some embodiments, the methods comprise: administering an effective amount of (S)-5-(1-(3,5-bis(trifluoromethyl)phenoxy)ethyl)cyclohexane-1,3-dione, or a suitable pharmaceutical salt thereof, to the subject to treat Alzheimer's disease in the subject. In some embodiments, the method treats memory loss in the subject. In some embodiments, the subject suffers from amyloid-beta oligomerization. In some embodiments, the subject is administered a daily dose of the compound of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values. In some embodiments, the compound is administered orally. In some embodiments, the methods further comprise administering a cholinesterase inhibitor and/or a N-methyl-D-aspartate receptor antagonist. In some embodiments, the method comprises administering the cholinesterase inhibitor, the cholinesterase inhibitor selected from galantamine, rivastigmine, and donepezil. In some embodiments, the method comprises administering the N-methyl-D-aspartate receptor antagonist memantine.

In another aspect of the current disclosure, methods of treating or preventing a disease or disorder associated with amyloid-beta oligomerization in a subject in need thereof are provided. In some embodiments, the methods comprise administering an effective amount of (S)-5-(1-(3,5-bis(trifluoromethyl)phenoxy)ethyl)cyclohexane-1,3-dione, or a suitable pharmaceutical salt thereof, to the subject to treat a disease or disorder associated with amyloid-beta oligomerization in the subject. In some embodiments, the disease or disorder is selected from Alzheimer's disease, cerebral amyloid angiopathy (CAA), inflammatory cerebral amyloid angiopathy, frontotemporal dementia and cerebral amyloidoma. In some embodiments, the method treats memory loss in the subject. In some embodiments, the subject is administered a daily dose of the compound of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values. In some embodiments, the compound is administered orally. In some embodiments, the methods further comprise administering a cholinesterase inhibitor and/or a N-methyl-D-aspartate receptor antagonist. In some embodiments, the method comprises administering the cholinesterase inhibitor, the cholinesterase inhibitor selected from galantamine, rivastigmine, and donepezil. In some embodiments, the method comprises administering the N-methyl-D-aspartate receptor antagonist memantine.

In another aspect of the current disclosure, methods of modulating amyloid beta oligomerization activity in a subject's brain are provided. In some embodiments, the methods comprise: administering an effective amount of (S)-5-(1-(3,5-bis(trifluoromethyl)phenoxy)ethyl)cyclohexane-1,3-dione, or a suitable pharmaceutical salt thereof, to the subject to modulate amyloid-beta oligomerization activity in the subject's brain. In some embodiments, the method inhibits formation of neuron binding amyloid beta oligomers. In some embodiments, the method promotes formation of non-binding amyloid beta oligomers. In some embodiments, the subject suffers from Alzheimer's disease. In some embodiments, the method treats memory loss in the subject. In some embodiments, the subject suffers from amyloid-beta oligomerization in the brain. In some embodiments, the subject is administered a daily dose of the compound of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values. In some embodiments, the compound is administered orally. In some embodiments, the methods further comprise administering a cholinesterase inhibitor and/or a N-methyl-D-aspartate receptor antagonist. In some embodiments, the method comprises administering the cholinesterase inhibitor, the cholinesterase inhibitor selected from galantamine, rivastigmine, and donepezil. In some embodiments, the method comprises administering the N-methyl-D-aspartate receptor antagonist memantine.

In another aspect of the current disclosure, methods for detecting candidate compounds that modulate amyloid-beta oligomerization in the presence of cells are provided. In some embodiments, the methods comprise: (i) culturing cells with amyloid-beta peptide in the presence and absence of a candidate compound and contacting control cells with amyloid beta peptide in the presence and absence of a control compound; (ii) detecting one or more parameters related to oligomerization of amyloid-beta in the cells of step (i); (iii) generating a test index by calculating a change in the one or more parameters between the cells cultured in the presence and absence of the candidate compound and generating a control index by calculating a change in the one or more parameters between the cells cultured in the presence and absence of the control compound; wherein the control compound is (S)-5-(1-(3,5-bis(trifluoromethyl)phenoxy)ethyl)cyclohexane-1,3-dione, or a pharmaceutically acceptable salt thereof, and wherein, if the value of the test index is equal to, or improved, as compared to the value of the control index, then the candidate compound modulates amyloid-beta oligomerization. In some embodiments, the cells are neurons, or are derived from neurons. In some embodiments, the cells are E18 hippocampal neurons. In some embodiments, the one or more parameters comprise detecting amyloid-beta oligomers (AbO) bound to the cells.

In another aspect of the current disclosure unit dosage packages are provided. In some embodiments, the unit dosage packages comprise: (i) (S)-5-(1-(3,5-bis(trifluoromethyl)phenoxy)ethyl)cyclohexane-1,3-dione, or a pharmaceutically acceptable salt thereof; and (ii) a cholinesterase inhibitor or an N-methyl-D-aspartate receptor antagonist. In some embodiments, the cholinesterase inhibitor is selected from: galantamine, rivastigmine, and donepezil. In some embodiments, the N-methyl-D-aspartate receptor antagonist is memantine.

In another aspect of the current disclosure, pharmaceutical compositions are provided. In some embodiments, the pharmaceutical compositions comprise: (i) (S)-5-(1-(3,5-bis(trifluoromethyl)phenoxy)ethyl)cyclohexane-1,3-dione, or a pharmaceutically acceptable salt thereof; (ii) a cholinesterase inhibitor or N-methyl-D-aspartate receptor antagonist; and (iii) a pharmaceutically acceptable carrier or excipient. In some embodiments, the cholinesterase inhibitor is selected from: galantamine, rivastigmine, and donepezil. In some embodiments, the N-methyl-D-aspartate receptor antagonist is memantine.

### BRIEF DESCRIPTION OF THE FIGURES

FIG. 1 shows the effect of NU-9 on binding of oligomers to neurons. Neurons were pre-treated with 3 μM NU-9 for 30 min, then treated with Aβ42 monomer for 24 hr. Cells were fixed with 3.7% formaldehyde and stained for AβOs (NU4, green), tau phosphorylated at Ser396 (pSer396, red) and mounted on slides before imaging. Analysis shows that the presence of NU-9 significantly decreased the number of AβOs bound at dendrites (p=0.001, 5 samples).

FIG. 2 shows neurons pre-treated with 3 μM NU-9 for 30 min, then treated with Aβ42 monomer for 26 hr. Cells were fixed with 3.7% formaldehyde and stained for AβOs (NU4, green), tau phosphorylated at Thr205 (pThr205, red), and nuclei (DAPI, blue) and mounted on slides before imaging. Analysis shows that the presence of NU-9 significantly decreased the number of AβOs bound at dendrites (p=0.0005, 13-15 samples).

FIG. 3. Neurons were pre-treated with 3 μM NU-9 for 30 min, then treated with Aβ42 monomer for 30 min. Cells were fixed with 3.7% formaldehyde and stained for AβOs (NU2, green), β-II tubulin (TUBB3, red), and nuclei (DAPI, blue) and mounted on slides before imaging.

FIG. 4. E18 hippocampal neurons were cultured until 21 div and pre-treated with 3, 15, or 30 μM NU-9 for 30 minutes, followed by treatment with 500 nM Aβ42 for 30 minutes. Cells were fixed with 3.7% formaldehyde and stained for AβOs (NU2, green), 3-III tubulin (red) and nuclei (DAPI, blue), and mounted on slides before imaging at 63×. More extensive suppression of AβOs was observed at higher concentrations of NU-9.

FIG. 5. A) NU-9 and Aβ were combined in F12 media, in the absence of cells, and incubated at 37° C. B) The concentration of AβOs was probed with NU2 antibody via dot blot after 24 hours in the conditions containing 3 μM NU-9 with or without Aβ. No significant difference was observed between the solution containing 3 μM NU-9 and Aβ and the solution with Aβ and vehicle.

FIG. 6. In dishes without cells, but in conditions that mimicked cell culture, 3 μM NU-9 and 500 nM Aβ42 for 30 minutes fixed with 3.7% formaldehyde and stained for AβOs (NU2, green) and mounted on slides before imaging at 63×. The presence of NU-9 did not significantly affect the number of AβOs bound to coverslips (p=0.5).

FIG. 7. NU-9 mitigates memory loss in older 5×FAD mice. Preliminary results obtained in 11 month old mice treated with NU-9 for 4 weeks. Memory performance was evaluated before and after treatment with vehicle or NU-9. All animals showed normal motor and exploratory behavior; not shown.

FIG. 8. NU-9 remains effective even when it is washed out prior to addition of Aβ monomer. Mature hippocampal neurons were treated with DMSO control (Control) or NU-9. Then, NU-9 or vehicle was washed out and Aβ was added (500 nM, 30 min). Cells were labelled with NU2 (green, anti-AβO) and MAP2 (red, anti-dendrite).

FIG. 9. The effect of NU-9 requires the presence of cells. Conditioned media was collected from mature hippocampal neuron. DMSO control (Control), Aβ monomer (Aβ), or NU-9 and then Aβ (NU-9+Aβ in cond. media) were added, then concentration of AβOs were analyzed by dot blot. Conditioned media was also collected from neurons treated with NU-9, then Aβ was added to this (Media from NU-9 in cells+Aβ). Aβ was not reduce in any treatment condition. Standard curve was created in media using 0-1000 fmol AβOs. The dot blot was labelled with NU2 (anti-AβO).

FIG. 10. NU-9 does not reduce AβO formation from Aβ monomer in cell-free solution; nor does conditioned media obtained from cells treated with NU-9. (Conditioned Media) A dot blot probed with NU2 was used to measure AβO abundance in solutions of conditioned media to which DMSO vehicle (control), Aβ, and NU-9 followed by Aβ were added for 30 min each. The bottom row corresponds to samples in which cells were treated with NU-9 for 30 min, then extracellular media was removed, and Aβ was added to the media for 30 min. The standard curve at the top was made from 0-1000 fmol preformed AβOs. (Fresh Media) A dot blot probed with NU2 was used to measure AβO abundance in solutions of culture media to which DMSO vehicle (control), Aβ, and NU-9 (3-300 μM) followed by Aβ were added for 30 min each. Each of these solutions was prepared in triplicate. The standard curve at the top was made from 0-1000 fmol preformed AβOs.

FIG. 11. NU-9 does not reduce binding of preformed AβOs to neurons. Hippocampal neurons were treated with 3 μM NU-9 or vehicle for 30 min, then with Aβ monomer or preformed AβOs for 24 h. NU-9 was able to reduce AβO binding in neurons treated with Aβ monomer, but not in neurons treated with preformed AβOs. Neurons were stained with NU4 (anti-AβO antibody) and puncta per process were quantified.

FIG. 12. NU-9 does not reduce total Aβ species. Mature hippocampal neurons were treated with NU-9 (3 μM, 30 min), then with Aβ monomer (500 nM, 30 min). These were labelled with 6E10 (anti-total Aβ, green) and MAP2 (anti-dendrite). Total number of Aβ particles per images as well as fluorescence intensity of Aβ signal (IntDen) was quantified).

FIG. 13. Lysosome inhibition prevents the effect of NU-9 on AβOs bound to synapses. Mature hippocampal neurons were treated for 30 min with 100 nM Bafilomycin A, then 30 min with 3 μM NU-9, and finally 30 min with 500 nM Aβ. Cell cultures were fixed and labelled with NU2 (anti-AβO antibody) and MAP2 (anti-dendrite antibody). Number of AβOs bound per micron along the dendrites were quantified using SynPAnal.

FIG. 14. Lysosome acidification as a potential mechanism for the effect of NU-9 on AβOs bound to synapses. Potential effects of NU-9 are shown in orange. NU-9 could activate endosomal uptake of Aβ monomer, endo-lysosomal trafficking of Aβ monomer and small oligomers. Alternately, it might activate acidification of the lysosome and release of non-binding oligomers, leading to reduced escape of large oligomers from less-acidic endo-lysosomal vesicles.

FIG. 15. Mature hippocampal neurons were pre-treated with vehicle or 3 μM NU-9 for 30 min, then vehicle, 200 nM, or 500 nM AβO for 30 min. Then cells were fixed and labelled using anti-MAP2 (green, dendrites), DAPI (blue, nucleus), and NU2 (red, AβOs). For both concentrations of AβO, NU-9 had no effect on binding to dendrites. The graph shows preliminary quantification puncta/μm dendrite for vehicle, 200 nM AβO, and 200 nM AβO+9 (analysis of 10 images/condition, *** indicates p<0.001).

FIG. 16. Lysosome inhibition prevents the effect of NU-9. Mature hippocampal neurons were pre-treated with vehicle or 100 nM Bafilomycin A for 30 min, followed by vehicle or 3 μM NU-9 for another 30 min, and last with vehicle or 500 nM Aβ monomer for a final 30 min. Cells were then fixed and labelled using anti-MAP2 (green, dendrites), DAPI (blue, nucleus), and NU2 (red, AβOs). As before, Bafilomycin A significantly prevented the effect of NU-9. The graph shows quantification of NU2 puncta/μm along the dendrite (analysis of 15 images/condition, *** indicates p<0.001, and ** indicates p<0.01).

FIG. 17. Cysteine cathepsin inhibition does not prevent the effect of NU-9. Mature hippocampal neurons were treated first with 10 μM E64 or vehicle for 24 h, then with 3 μM NU-9 for 30 min, and finally with 500 nM Aβ monomer for the last 30 min. Then, cells were fixed and labelled with anti-MAP2 (red, dendrites), DAPI (blue, nucleus), and NU2 (green, AβOs). The graph shows quantification of puncta/μm dendrite (20 images analyzed/condition, *** indicates p<0.001 and ** indicates p<0.01).

FIG. 18. Mature hippocampal neurons were treated with vehicle or 100 nM Bafilomycin A for 30 min, then labelled with Lysotracker DND-99 (red) at 75 nM for 30 min or 50 nM, 2 h as shown. Both conditions yielded clear labelling of small structures. The 75 nM, 30 min treatment yielded a brighter signal.

FIG. 19. Mature hippocampal neurons were treated first with 3 μM 9 for 30 min, and then with A) 30 or B) 200 nM LC AβOs for 30 min. Then, cells were fixed and labelled with anti-MAP2, DAPI, and NU2 (AβOs). The graphs show quantification of puncta/μm dendrite (30 images analyzed/condition, *** indicates p<0.001, ** p<0.01, * p<0.05).

FIG. 20. A) Mature cultures of E18 hippocampal neurons were pre-treated for 30 min with 3 μM NU-9, followed by addition of 500 nM Aβ for 30 min. B) The graph shows quantification of puncta/μm dendrite (30 images analyzed/condition).

FIG. 21. Addition of monomeric Aβ₁₋₄₂ (500 nM) to mature E18 hippocampal neurons for 30 min resulted in production of unbound, extracellular AβOs, detected by dot blot. 30-minute pre-treatment with 3 μM NU-9 did not significantly reduce the number of unbound, extracellular AβOs bound to dendrites, in either supernatant (p=0.6) or uncentrifuged solution (p=0.3). 3 bioreplicates were carried out for each condition, and each was spotted in duplicate on the dot blot.

FIG. 22. A) Representative images of AβOs (green, NU2) bound to dendrites (red, MAP2) of mature hippocampal neurons to which were applied 500 nM Aβ42, with or without 30-min pre-treatment with 3 μM NU-9 and 30-min pre-treatment 100 nM MG132, and B) quantification of AβO puncta per μm along the dendrites, which was conducted using ImageJ, n=30 images/condition.

FIG. 23. A) Representative images of AβOs (green, NU2) bound to dendrites (red, MAP2) of mature hippocampal neurons to which were applied 500 nM Aβ42, with or without 30-min pre-treatment with 3 μM NU-9 and 1 h pre-treatment 1 μM vacuolin-1. B) is quantification of AβO puncta per μm along the dendrites, which was conducted using ImageJ, n=30 images/condition.

FIG. 24. Mature E18 hippocampal neurons were treated 3 μM NU-9, and subsequently labelled with lysotracker. A) total immunofluorescence (integrated density), B) mean intensity, C) average size of acidic compartments, D) number of acidic compartments. These values were calculated using ImageJ from 10 images across two coverslips of neurons, and similar trends were observed for another replicate in a different cell culture.

FIG. 25. A) Mature E18 hippocampal neurons were treated first with 10 μM CA-074 or vehicle for 24 h, then with 3 μM 9 for 30 min, and finally with 500 nM Aβ monomer for the last 30 min. Then, cells were fixed and labelled with anti-MAP2 (green, dendrites), DAPI (blue, nucleus), and NU2 (red, AβOs). B) The graph shows quantification of puncta/μm dendrite (30 images analyzed/condition, *** indicates p<0.001, ** p<0.01, * p<0.05).

FIG. 26. A) 0.0003-150 μM NU-9 was combined with purified cathepsin L enzyme in the presence of the cathepsin L substrate Z-FR-AMC. Cathepsin L enzyme activity was measured by the rate of 7-AMC production by fluorescence. Percent change in rate was quantified in the graph. The standard inhibitor Z-FY-CHO reduced cathepsin L activity over the same concentration range (not shown). B) Mature hippocampal neurons were treated with NU-9 for 30 minutes, then Magic Red was used to monitor intracellular cathepsin L activity. Cells were treated in 3 wells, and 9-22 images were analyzed per condition.

FIG. 27. Inhibition of cathepsin L prevents AβO accumulation, mimicking the effect of NU-9. Neurons were pre-treated first for 1 h with 10 μM Cathepsin L inhibitor (Cayman), then for 30 minutes with 3 μM NU-9, and finally for 30 minutes to 500 nM Aβ Then, cells were fixed and labelled with anti-MAP2 (green, dendrites), DAPI (blue, nucleus), and NU2 (red, AβOs). The graph shows quantification of puncta/μm dendrite.

FIG. 28. A) 0.0003-30 μM NU-9 was combined with purified cathepsin B enzyme in the presence of the cathepsin B substrate Z-RR-AMC. Cathepsin B enzyme activity was measured by the rate of 7-AMC production by fluorescence. Percent change in rate with NU-9 was quantified in the graph. The standard cathepsin B inhibitor CA-074 reduced cathepsin L activity over the same concentration range (not shown). B) Mature hippocampal neurons were treated with NU-9 for 30 minutes, then Magic Red was used to monitor intracellular cathepsin L activity. Cells were treated in 3 wells.

FIG. 29. Calpain inhibition may mimic the effect of NU-9. E18 hippocampal neurons at 21 div were treated with 10 μM MDL-28170 for 30 min, then 3 μM NU-9 for 30 min, and finally 500 nM Aβ for 30 min. Cells were labelled by immunofluorescence for AβOs, with NU2, and neuronal dendrites, with MAP2. Number of AβOs per micron along the dendrites were quantified using ImageJ for 30 images in each condition, and data were analyzed using Prism.

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with amyloid beta activity and/or expression. A subject in need thereof may include a subject having a disease or disorder that is characterized by oligomerization or aggregation of amyloid beta. A “subject in need thereof” as utilized herein may include, but is not limited to a subject in need of treatment of Alzheimer's disease, cerebral amyloid angiopathy (CAA), inflammatory cerebral amyloid angiopathy, and cerebral amyloidoma.

The term “subject” may be used interchangeably with the terms “individual” and “patient” and includes human and non-human mammalian subjects.

The disclosed compounds, pharmaceutical compositions, and methods may be utilized to treat and/or prevent diseases and disorders associated with amyloid beta oligomerization which may include, but are not limited to Alzheimer's disease, cerebral amyloid angiopathy (CAA), inflammatory cerebral amyloid angiopathy, and cerebral amyloidoma.

The disclosed compounds may be utilized to modulate the biological activity of amyloid beta, including modulating the oligomerization activity of amyloid beta.

Amyloid beta refers to proteins derived from the cleavage of amyloid beta precursor protein (APP) by beta secretase and gamma secretase enzymes. Amyloid beta peptides may be between 36 and 43 amino acids in length.

Amyloid beta is produced by proteolytic processing of amyloid precursor protein (APP) by, for example, alpha and gamma secretases. The following amino acid sequence is an example of the sequence of APP (SEQ ID NO:1):

MLPGLALLLL AAWTARALEV PTDGNAGLLA EPQIAMFCGR 40
LNMHMNVQNG KWDSDPSGTK TCIDTKEGIL QYCQEVYPEL 80
QITNVVEANQ PVTIQNWCKR GRKQCKTHPH FVIPYRCLVG 120
EFVSDALLVP DKCKFLHQER MDVCETHLHW HTVAKETCSE 160
KSTNLHDYGM LLPCGIDKFR GVEFVCCPLA EESDNVDSAD 200
AEEDDSDVWW GGADTDYADG SEDKVVEVAE EEEVAEVEEE 240
EADDDEDDED GDEVEEEAEE PYEEATERTT SIATTTTTTT 280
ESVEEVVREV CSEQAETGPC RAMISRWYFD VTEGKCAPFF 320
YGGCGGNRNN FDTEEYCMAV CGSAMSQSLL KTTQEPLARD 360
PVKLPTTAAS TPDAVDKYLE TPGDENEHAH FQKAKERLEA 400
KHRERMSQVM REWEEAERQA KNLPKADKKA VIQHFQEKVE 440
SLEQEAANER QQLVETHMAR VEAMLNDRRR LALENYITAL 480
QAVPPRPRHV FNMLKKYVRA EQKDRQHTLK HFEHVRMVDP 520
KKAAQIRSQV MTHLRVIYER MNQSLSLLYN VPAVAEEIQD 560
EVDELLQKEQ NYSDDVLANM ISEPRISYGN DALMPSLTET 600
KTTVELLPVN GEFSLDDLQP WHSFGADSVP ANTENEVEPV 640
DARPAADRGL TTRPGSGLIN IKTEEISEVK MDAEFRHDSG 680
YEVHHQKLVF FAEDVGSNKG AIIGLMVGGV VIATVIVITL 720
VMLKKKQYTS IHHGVVEVDA AVTPEERHLS KMQQNGYENP 760
TYKFFEQMQN 770

Therefore, it will be clear to one of skill in the art that several possible isoforms of amyloid beta, produced by proteolytic processing from APP, may be found in subjects in need of treatment and/or prevention for a disease or disorder associated with amyloid beta oligomerization. The following are amino acid sequences for non-limiting examples of amyloid beta isoforms.

Amyloid beta 1-40 (aa 672-711 of APP) has the following amino acid sequence (SEQ ID NO:2):

DAEFRHDSGY EVHHQKLVFF AEDVGSNKGA IIGLMVGGVV 40

Amyloid beta 1-42 (aa 672-713 of APP) has the following amino acid sequence (SEQ ID NO:3):

DAEFRHDSGY EVHHQKLVFF AEDVGSNKGA IIGLMVGGVV IA 42

Amyloid beta may include N-terminal truncations, such as an N-terminal truncation of the first two amino acid residues, so called N3 amyloid beta such as amyloid beta (3-40) or amyloid beta (3-42). Amyloid beta may include pyroglutamic acid residues (pE) which replace glutamic acid residues. In some embodiments, Amyloid beta includes an N-terminal truncation of the first two amino acids and a pyroglutamic acid residue (pE) at the third position (N3pE), now the first position. Such amyloid beta may have a sequence described as (SEQ ID NO:4):

EFRHDSGYEV HHQKLVFFAE DVGSNKGAII GLMVGGVVIA 40

The various isoforms of amyloid-beta (Aβ) have different propensities for aggregation. Aβ pools may be characterized by three major groups of Aβ assemblies: monomers, soluble oligomers, and insoluble fibrils. Each pool may encompass multiple structures based on various organizations. The term “amyloid beta oligomerization” refers to the formation of soluble oligomers. Such soluble oligomers may be organized into different structures ranging from two or more Aβ, e.g., dimers, trimers, tetramers, pentamers, decamers, dodecamers and so forth. Soluble oligomers may also include, e.g., Aβ-derived diffusible ligands (ADDLs) and Aβ*56.

Toxic soluble oligomers are distinct from the monomers or higher aggregates, such as fibrils, and were identified in AD brains. There is an inverse correlation between the size of Aβ assemblies and the potency of their exerted toxicity. As the size of the oligomeric assembly increases, its deleterious effects decrease. Aβ dimers have been shown to assemble forming a more stable structure of higher molecular weight, termed protofibrils which are neurotoxic. Thus, dimeric units of Aβ have been considered to be an important entity providing the building blocks for the toxic aggregates. Thus, Aβ oligomerization includes the formation of toxic amyloid-beta oligomers (AβOs).

AβOs are deleterious to neurons and may be the causative agent for neurodegeneration observed in, e.g., Alzheimer's disease. Cells, e.g., cultured neurons, that are cultured or contacted with Aβ develop AβOs bound to the surface of the cells. However, the inventors discovered that the compound NU-9 modulates Aβ oligomerization activity.

As used herein, “modulate”, with regard to Aβ oligomerization activity, refers to the effect on Aβ aggregation. Modulation of Aβ may refer to a modulation of the Aβ pools (e.g., in the relative proportion between Aβ monomers, soluble oligomers, and insoluble fibrils), modulation of the soluble oligomers (e.g., in the relative proportion between dimers, trimers, tetramers, pentamers, decamers, and so forth), modulation of the organizational structure of soluble oligomers, or any combination thereof.

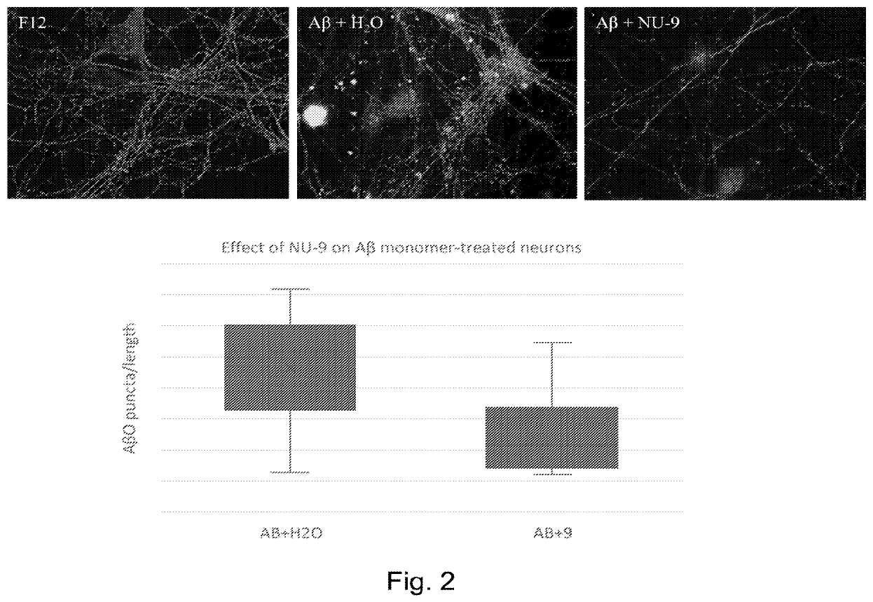

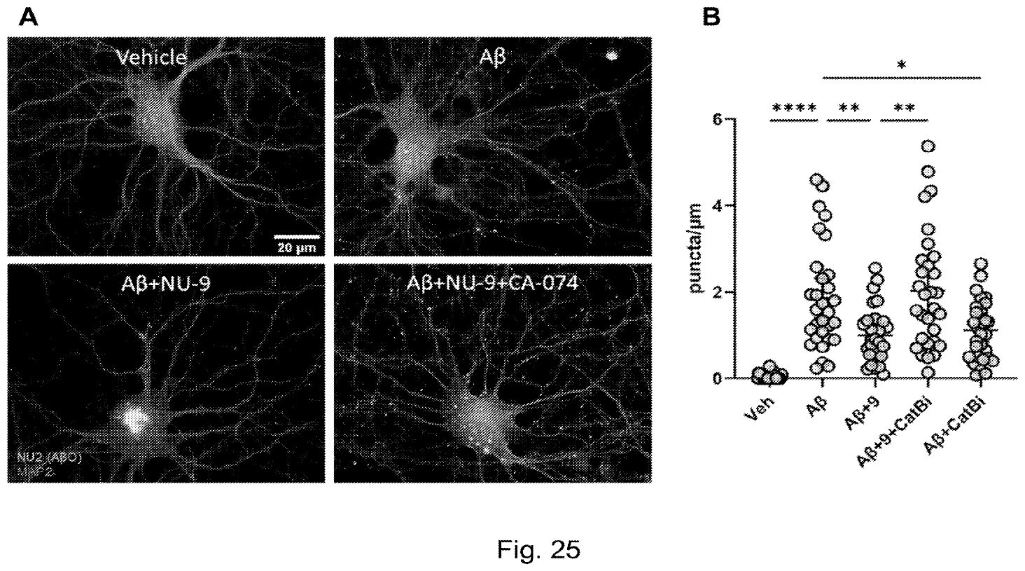

As demonstrated in the Examples, NU-9 effectively reduced the number of pathological species of AβOs that are produced from Aβ that are capable of binding to the surface of cells (FIG. 2). NU-9 also promoted formation of non-binding oligomers. The inventors also discovered that NU-9 acts through a lysosome-dependent mechanism to reduce the formation of toxic AβOs (FIGS. 25-29). Thus, in some embodiments, the methods include inhibiting the formation of neuron binding AβOs or toxic AβOs.

### Methods of Use

In one aspect of the current disclosure, methods of treating Alzheimer's disease in a subject in need thereof are provided. In some embodiments, the methods comprise administering an effective amount of NU-9, or a suitable pharmaceutical salt thereof, to the subject to treat Alzheimer's disease in the subject.

In another aspect of the current disclosure, methods of treating or preventing a disease or disorder associated with amyloid-beta oligomerization in a subject in need thereof are provided.

In another aspect of the current disclosure, methods of modulating amyloid beta oligomerization activity in a subject's brain are provided. In some embodiments, the methods comprise: administering an effective amount of NU-9, or a suitable pharmaceutical salt thereof, to the subject to modulate amyloid-beta oligomerization activity in the subject's brain.

In some embodiments, the methods of the instant disclosure further comprise administering at least one other compound to the subject selected from a cholinesterase inhibitor and an N-methyl-D-aspartate (NMDA) receptor antagonist. In some embodiments, the cholinesterase inhibitor is selected from: galantamine, rivastigmine, and donepezil. In some embodiments, the NMDA receptor antagonist is memantine.

Disclosed are compounds, pharmaceutical compositions comprising the compounds, and methods of using the compounds and pharmaceutical compositions for treating and/or preventing a disease or disorder associated with amyloid beta biological activity in a subject in need thereof. The disclosed compounds may include cyclohexane 1,3-diones, such as NU-9 and pharmaceutically acceptable salts thereof, that inhibit one or more biological activities of amyloid beta, such as amyloid beta oligomerization. As such, the disclosed compounds and pharmaceutical compositions may be utilized in methods for treating a subject having or at risk for developing a disease or disorder that is associated with amyloid beta activity which may be disease and disorders associated with amyloid beta oligomerization.

The disclosed compounds include cyclohexane 1,3-diones. Cyclohexane 1,3-diones and methods for synthesizing cyclohexane 1,3-diones are disclosed in the art. (See e.g., Zhang et al., “Chiral Cyclohexan 1,3-diones as Inhibitors of Mutant SOD1-Dependent Protein Aggregation for the Treatment of ALS,” ACS Medic. Chem. Lett., 20021, 3, 584-587, the content of which is incorporated herein by reference in its entirety). The disclosed compounds for uses as disclosed herein may include, but are not limited to (S)-5-(1-(3,5-bis(trifluoromethyl)phenoxy)ethyl)cyclohexane-1,3-dione (NU-9).

In some embodiments, the disclosed methods may be performed in order to treat and/or prevent a disease or disorder is selected from, but not limited to, Alzheimer's disease, cerebral amyloid angiopathy (CAA), inflammatory cerebral amyloid angiopathy, frontotemporal dementia and cerebral amyloidoma. In some embodiments, the disclosed methods may be performed in order to treat and/or prevent one or more symptoms of a disease or disorder associated with amyloid beta activity.

The disclosed methods may be performed in order to treat and/or prevent memory loss in a subject. For example, the disclosed methods may be performed in order to treat and/or prevent memory loss in a subject that is associated with amyloid beta oligomerization and impaired neuronal function.

In the disclosed methods, the subject may be administered an effective amount of the disclosed compounds in order to treat and/or prevent amyloid beta oligomerization in the subject. In some embodiments of the disclosed methods, the subject is administered a daily dose of the disclosed compounds of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values.

In the disclosed methods, the compounds and pharmaceutical compositions may be administered to the subject by any suitable route in order to delivery an effective amount of the disclosed compounds to a site in a subject that is exhibiting amyloid beta oligomerization or to a site in the subject that is at risk for incurring amyloid beta oligomerization, such as the brain of the subject. In some embodiments, the compounds and pharmaceutical compositions are administered through an oral route.

### Pharmaceutical Compositions

In another aspect of the current disclosure, pharmaceutical compositions are provided. In some embodiments, the pharmaceutical compositions comprise: (i) NU-9, or a pharmaceutically acceptable salt thereof, and (ii) a pharmaceutically acceptable carrier or excipient.

In some embodiments, the pharmaceutical compositions combine NU-9 with another compound for use in the treatment of Alzheimer's disease. In some embodiments, the pharmaceutical compositions comprise: (i) NU-9, or a pharmaceutically acceptable salt thereof, (ii) a cholinesterase inhibitor or N-methyl-D-aspartate receptor antagonist; and (iii) a pharmaceutically acceptable carrier or excipient. In some embodiments, the cholinesterase inhibitor is selected from: galantamine, rivastigmine, and donepezil. In some embodiments, the N-methyl-D-aspartate receptor antagonist is memantine.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that inhibits the biological activity of amyloid beta may be administered as a single compound or in combination with another compound inhibits the biological activity of amyloid beta or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and arylalkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of amyloid beta. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of amyloid beta.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 1000 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Unit Dosage Packages

In another aspect of the current disclosure, unit dosage packages are provided. Unit dosage packages comprise a first unit dosage including a first drug, such as NU-9 or a pharmaceutically acceptable salt thereof. Unit dosage packages may also comprise a second unit dosage comprising a second drug. The unit dosage package may comprise a container or label indicating the name, strength, control number, expiration date, administration instructions, or any combination thereof for the one or more drugs in the unit dosage package.

In some embodiments, the unit dosage packages comprise NU-9, or a pharmaceutically acceptable salt thereof, for treating amyloid-beta oligomerization, e.g., as observed in Alzheimer's disease. In some embodiments, the unit dosage packages comprise: (i) NU-9, or a pharmaceutically acceptable salt thereof, and (ii) a cholinesterase inhibitor or an N-methyl-D-aspartate receptor antagonist. In some embodiments, the cholinesterase inhibitor is selected from: galantamine, rivastigmine, and donepezil. In some embodiments, the N-methyl-D-aspartate receptor antagonist is memantine.

### Methods of Detecting a Candidate Compound

In another aspect of the current disclosure, methods of detecting a candidate compound that modulates amyloid-beta oligomerization in the presence of cells are provided. In some embodiments, the methods comprise: (i) culturing cells with amyloid-beta peptide in the presence and absence of a candidate compound and contacting control cells with amyloid beta peptide in the presence and absence of NU-9, or a pharmaceutically acceptable salt thereof, (ii) detecting one or more parameters related to oligomerization of amyloid-beta in the cells of step (i); (iii) generating a test index by calculating a change in the one or more parameters between the cells cultured in the presence and absence of the candidate compound and generating a control index by calculating a change in the one or more parameters between the cells cultured in the presence and absence of the control compound; wherein, if the value of the test index is equal to, or improved, as compared to the value of the control index, then the candidate compound modulates amyloid-beta oligomerization.

As used herein, use of the phrase “improved as compared to the value of the control index” refers to the test index having a greater value than the control index in a situation where the greater value is associated with a beneficial effect or a reduced value than the control index in a situation where the reduced value is associated with a beneficial effect. The phrase reflects that some values associated with a beneficial effect may be increased in the presence of the control compound, e.g., production of non-pathological Aβ species, or decreased in the presence of the control compound, e.g., production of pathological AβO species.

In some embodiments, the one or more parameters related to oligomerization includes detecting the quantity, or relative quantity, of AβOs on the surface of the cells. The quantity of AβOs on the surface of the cells may be determined through various techniques known in the art, e.g., fluorescent microscopy, flow cytometry, or other means known in the art to label a protein and detect said protein on the surface of cells, e.g., radioisotope labeling, gold-labelling in conjunction with transmission electron microscopy, etc.

In some embodiments, the cells are derived from neural progenitor cells (NPCs). NPCs are the progenitor cells of the central nervous system that give rise to many of the neuronal or glial cells types that populate the CNS. Cells derived from NPCs, include, without limitation, neurons, cells isolated from primary neuronal tissue, microglia, glial cells, astrocytes, oligodendrocytes, and the like, or are derived from neurons. As used herein, “derived from neurons” refers to any cell that was originally isolated from neuronal tissue, transformed from a primary neuronal cell to express one or more immortalization factors, e.g., hTERT, or an immortal cell line discovered in neuronal tissue. In some embodiments, the cells are E18 hippocampal neurons.

### EXAMPLES

The following Examples are illustrative and should not be interpreted to limit the scope of the claimed subject matter.

### Example 1—Effect of NU-9 on ABO Binding to AB42-Treated Neurons

In neurons treated with amyloid beta peptide, (S)-5-(1-(3,5-bis(trifluoromethyl)phenoxy)ethyl)cyclohexane-1,3-dione (NU-9) reduces the number of bound amyloid beta oligomers. In an Alzheimer's disease model mice, this compound mitigated memory loss. NU-9 disrupts the accumulation of toxic amyloid beta oligomers, as well as other aggregated species.

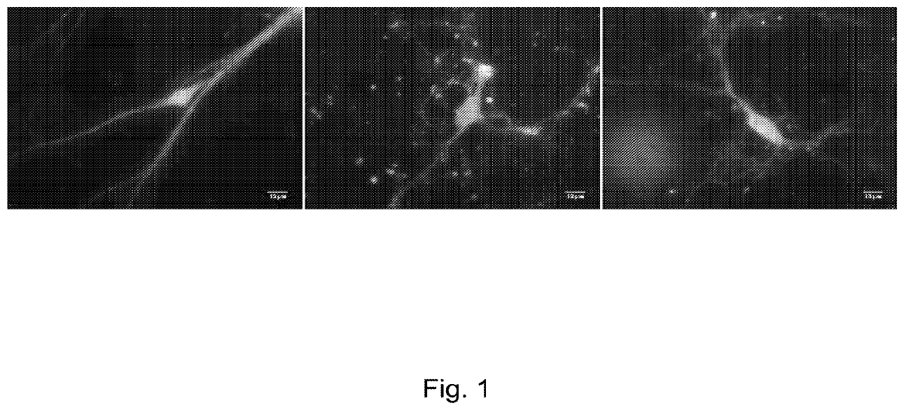

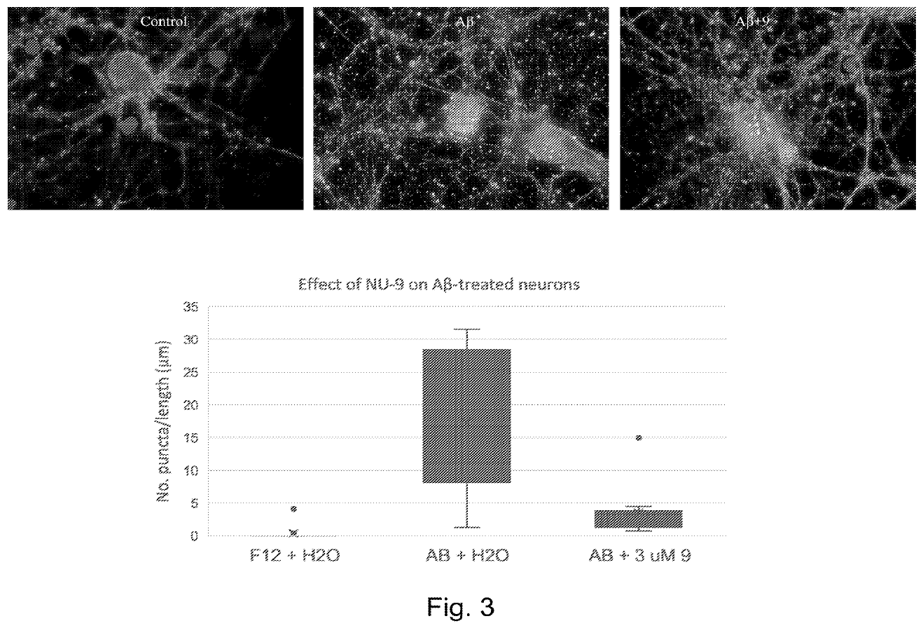

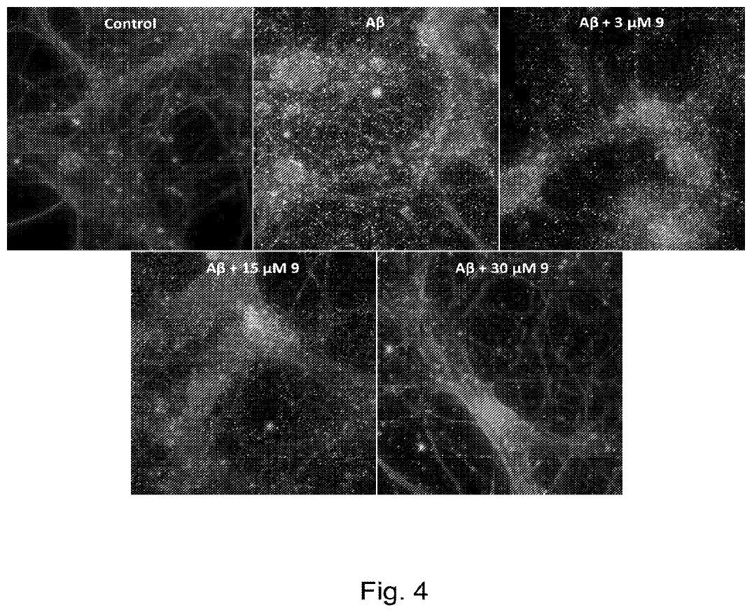

NU-9 reduces the number of amyloid beta oligomers bound to neurites. Primary rat hippocampal neurons in cell culture were pre-treated with NU-9 for 30 minutes and then treated with amyloid beta 1-42 (Aβ) monomer. After 24 hours (FIGS. 1 and 2) or 30 minutes (FIG. 3) of Aβ monomer application, pre-treatment with this compound dramatically decreased the number of Aβ oligomers (AβOs) bound to neurons after 24 hours. To characterize the dose-response effect of NU-9 on AβO formation, cells were pretreated with increasing doses of NU-9 (3, 15, and 30 μM), and then treated with Aβ for 30 minutes. In a visual analysis of images, Aβ was reduced more extensively at higher doses of NU-9 (FIG. 4).

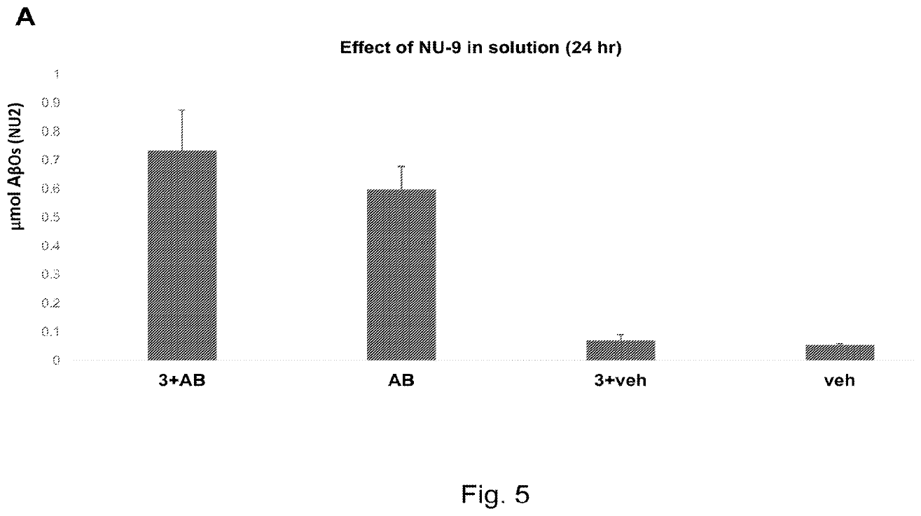

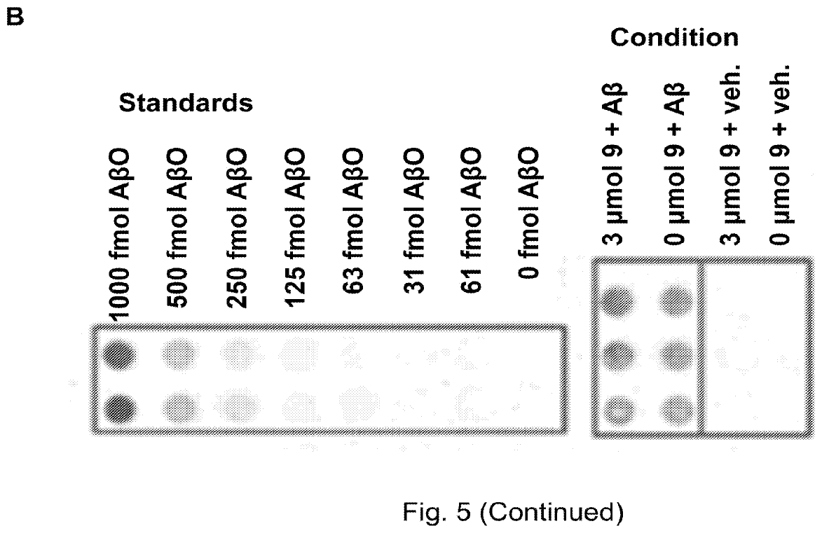

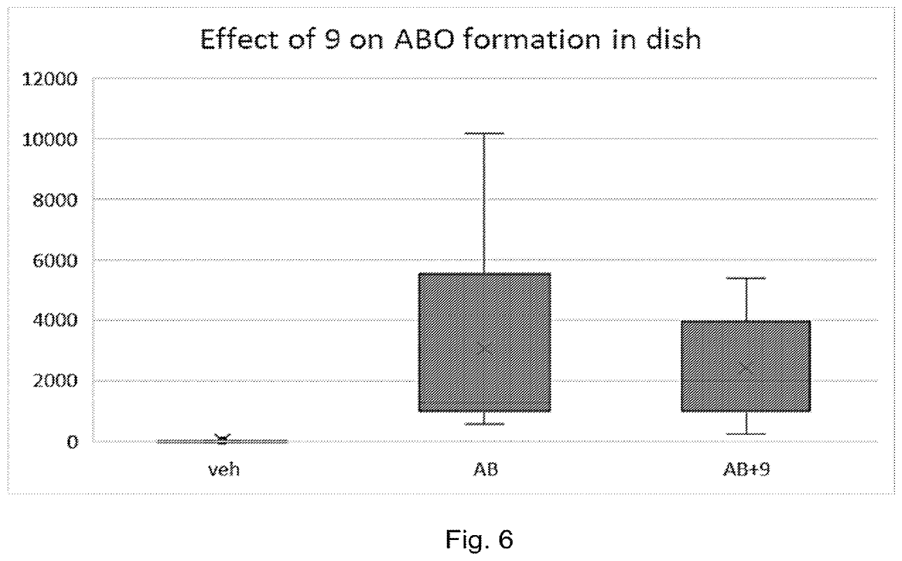

In the absence of cells, NU-9 has no effect on AβO formation. When NU-9 was mixed with Aβ monomer for 24 hours in the absence of cells, no change in AβO formation was observed (FIG. 5). This indicates that NU-9 acts via a cell-based mechanism. Next, a follow-up experiment was conducted using conditions that more closely mimicked those of cell culture. Coverslips were coated, as usual for cell culture, with poly-D-lysine, and then placed into culture dishes with media. No cells were added. Then 3 μM NU-9 and 500 nM AβOs were added for 30 min. In this assay, the previous result was replicated. Without cells, NU-9 did not affect AβO formation (FIG. 6).

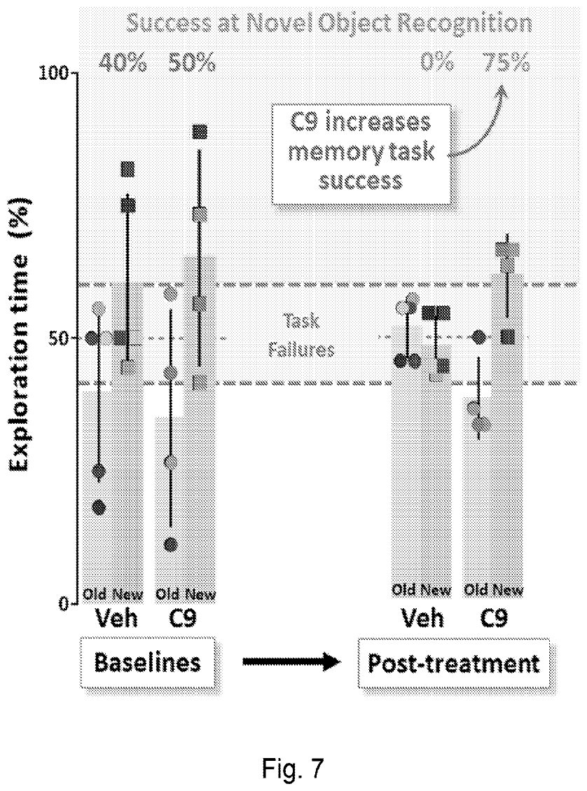

In a preliminary experiment using a mouse model of Alzheimer's disease, NU-9 protected against memory loss. Twelve 5×FAD mice were bred on the slow-onset background. These animals received either vehicle control or NU-9 by gavage (20 mg/kg) daily for one month. In the control group, all animals failed the memory task, showing no preference for the novel object. In the treated group, three of the four mice were protected from memory loss, showing a preference for novel object. (FIG. 7).

### Sources Cited

Kayed, R.; Head, E.; Thompson, J. L.; McIntire, T. M.; Milton, S. C.; Cotman, C. W.; Glabe, C. G., Common Structure of Soluble Amyloid Oligomers Implies Common Mechanism of Pathogenesis. Science 2003, 300 (5618), 486.
Balducci, C.; Beeg, M.; Stravalaci, M.; Bastone, A.; Sclip, A.; Biasini, E.; Tapella, L.; Colombo, L.; Manzoni, C.; Borsello, T.; Chiesa, R.; Gobbi, M.; Salmona, M.; Forloni, G., Synthetic amyloid-beta oligomers impair long-term memory independently of cellular prion protein. Proc. Natl. Acad. Sci. 2010, 107 (5), 2295-2300.
Lambert, M. P.; Barlow, A. K.; Chromy, B. A.; Edwards, C.; Freed, R.; Liosatos, M.; Morgan, T. E.; Rozovsky, I.; Trommer, B.; Viola, K. L.; Wals, P.; Zhang, C.; Finch, C. E.; Krafft, G. A.; Klein, W. L., Diffusible, nonfibrillar ligands derived from Aβ1-42 are potent central nervous system neurotoxins. Proc. Natl. Acad. Sci. 1998, 95 (11), 6448-6453.
Trippier, P. C.; Zhao, K. T.; Fox, S. G.; Schiefer, I. T.; Benmohamed, R.; Moran, J.; Kirsch, D. R.; Morimoto, R. I.; Silverman, R. B., Proteasome activation is a mechanism for pyrazolone small molecules displaying therapeutic potential in amyotrophic lateral sclerosis. ACS Chem Neurosci 2014, 5 (9), 823-9.
Leeman, D. S.; Hebestreit, K.; Ruetz, T.; Webb, A. E.; McKay, A.; Pollina, E. A.; Dulken, B. W.; Zhao, X.; Yeo, R. W.; Ho, T. T.; Mahmoudi, S.; Devarajan, K.; Passegue, E.; Rando, T. A.; Frydman, J.; Brunet, A., Lysosome activation clears aggregates and enhances quiescent neural stem cell activation during aging. Science 2018, 359 (6381), 1277.

### Example 2—Investigation into the Mechanism by which NU-9 Affects ABO Formation and Binding to Neurons

Adding NU-9 (3 μM, 30 min) to neurons prior to treatment with Aβ monomer (500 nM, 30 min or 24 h) caused a marked reduction in AβOs at synapses compared to treatment with Aβ monomer alone. This result was replicated over three separate experiments. The decrease was 81% (p=0.0005). We further characterized the effect of NU-9, to narrow down potential candidate mechanisms for this effect.

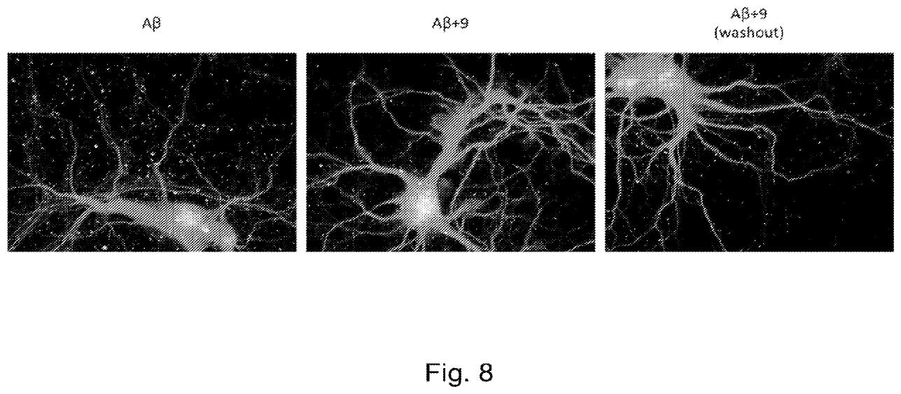

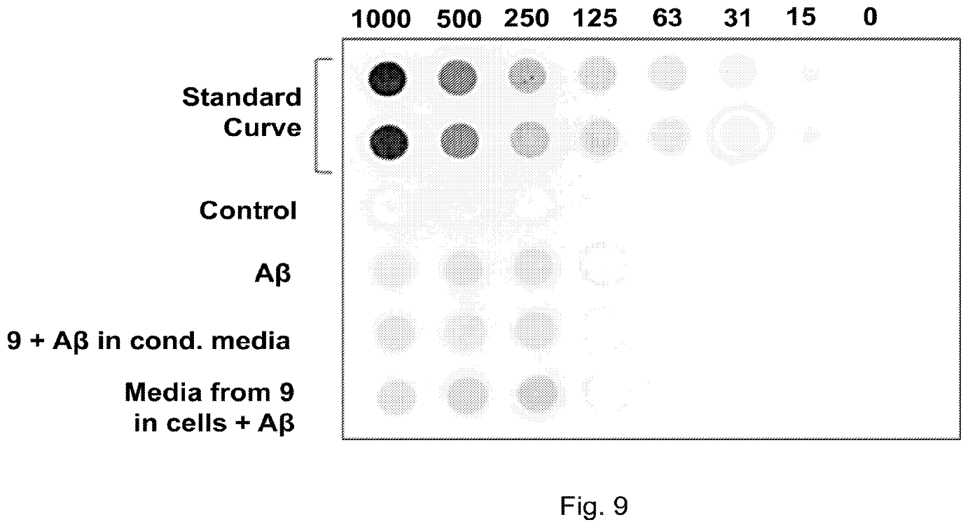

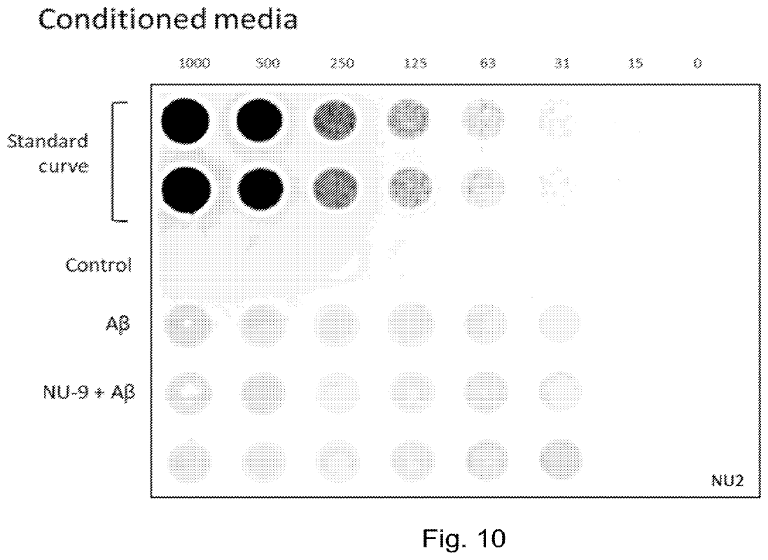

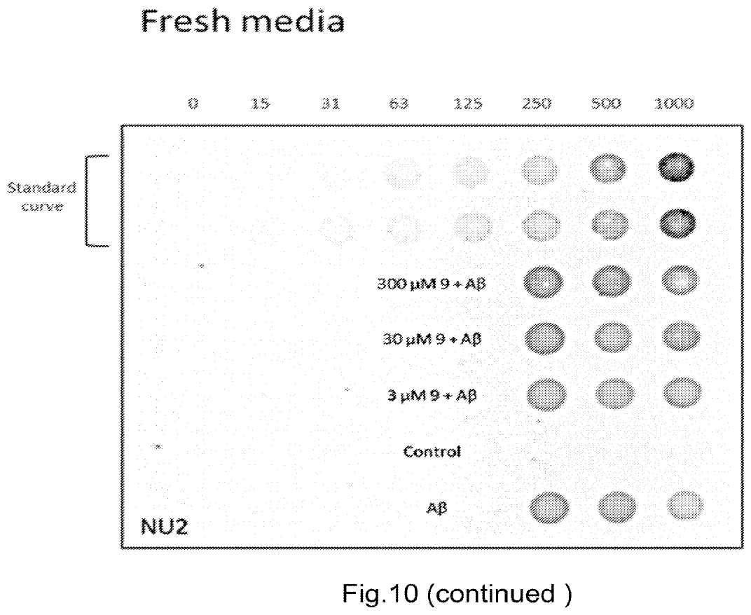

First, we expanded on previous experiments demonstrating that the effect of NU-9 requires cells. We observed that pre-treatment of cells with NU-9 leads to a reduction in AβOs bound to dendrites, even when NU-9 is washed out prior to addition of Aβ monomer (FIG. 8). Additionally, we collected conditioned media produced by untreated cells and found that adding NU-9 (3 μM, 30 min) to the media did not reduce the number of AβOs formed from Aβ monomer (500 nM, 30 min) (FIG. 9, p=0.4). We also collected conditioned media produced by cells treated with 3 μM NU-9 for 30 min and found that this media did not reduce the number of AβOs formed from Aβ monomer in the absence of cells (p=0.06). In dot blot analysis, addition of NU-9 to Aβ monomer did not reduce ABO formation directly in fresh (p 0.85 for 3 μM, 0.18 for 30 μM, 0.13 for 300 μM) media or conditioned media (p 0.88 NU-9+Aβ, 0.54 for media from NU-9 in cells+Aβ) (FIG. 10). Based on this experiment, we concluded that NU-9 does not prevent extracellular AβO formation. Interestingly, NU-9 neither acts by direct interaction with Aβ, nor by inducing secretion of AβO-degrading proteases, nor by inducing secretion of chaperones to stabilize Aβ monomer. Instead, NU-9 exerts an effect at the cell membrane or intracellularly.

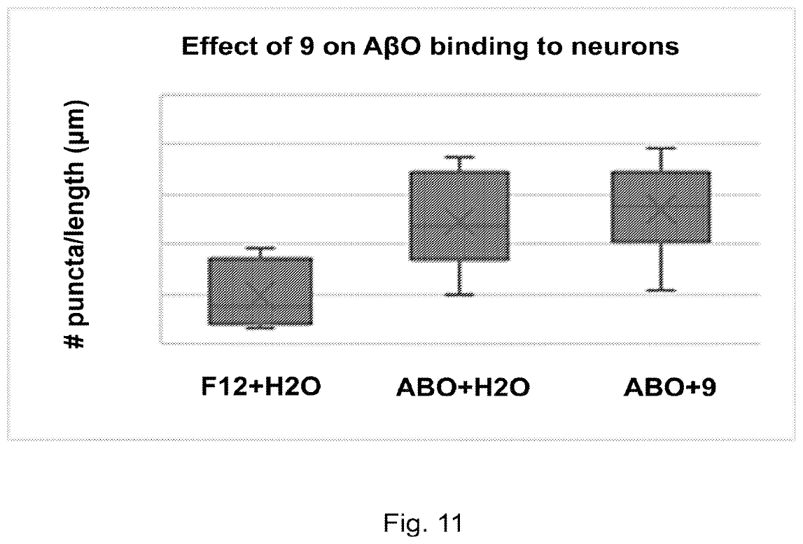

A previous publication by Pitt et al. showed that astrocytes confer protection against AβOs to neurons by releasing insulin and insulin-like growth factor, which cause the release of AβOs from neuronal synapses¹. However, we previously found that while NU-9 reduces AβO binding to neurons treated with Aβ monomer, it does not reduce binding in neurons treated with premade AβOs (FIG. 11). This suggests that NU-9 does not prevent AβO binding by inducing AβO release. In addition, it suggests that NU-9 does not act by direct interference with AβO binding, nor by inducing internalization of binding receptors.

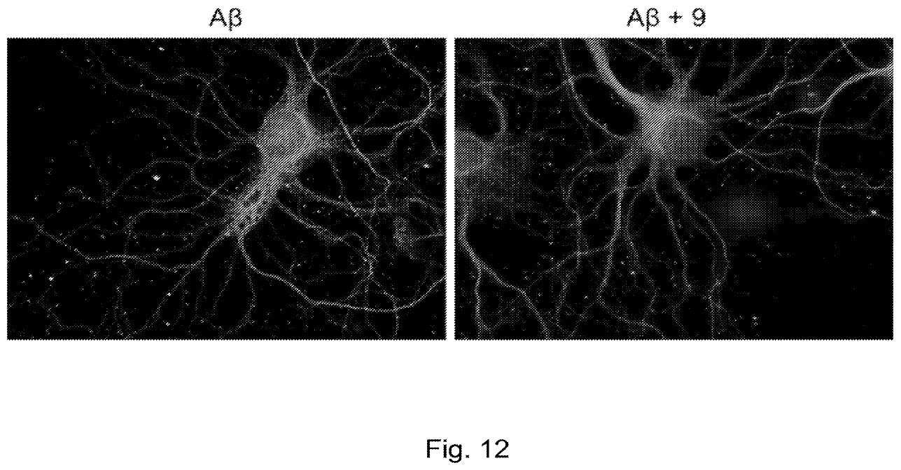

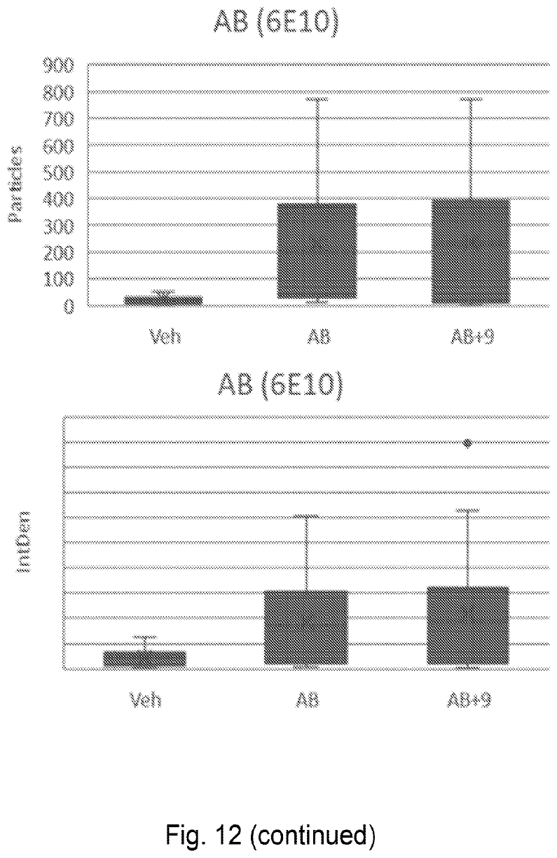

Next, we expanded upon results that indicate that NU-9 does not activate proteasomal degradation of AβOs. Previously, we found that proteasome inhibition does not increase the number of AβOs produced by Aβ monomer-treated neurons, supporting the hypothesis that proteasome activation may not be the mechanism by which NU-9 acts. To follow up on this, we treated neurons with NU-9 (3 μM, 30 min) followed by Aβ monomer (500 nM, 30 min) and attempted to observe total Aβ species by dot blot. However, the total Aβ antibody (6E10) showed a nonspecific interaction with cell culture media. So, instead of using dot blot, we used immunofluorescence, labelling treated neurons for total Aβ species. Using this protocol, we were able to avoid interaction of the antibody with cell culture media, allowing for specific labelling. Using immunofluorescence, we found that NU-9 does not reduce total Aβ species observable in neuronal cultures (FIG. 12, p=0.9 for particles, p=0.4 for IntDen).

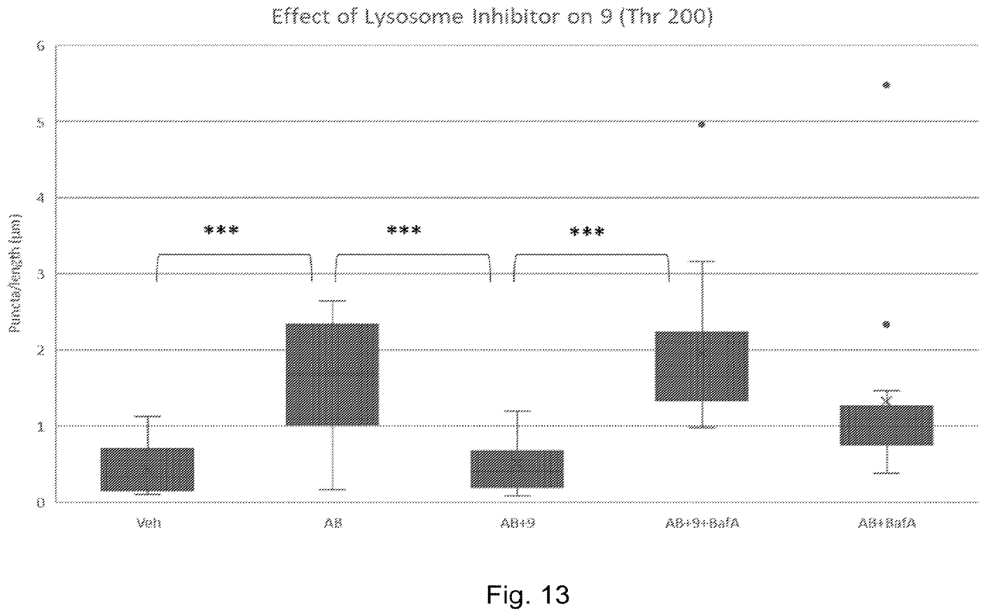

Effect of lysosome inhibition on effectiveness of NU-9. Cells were treated first with lysosome inhibitor (100 nM bafilomycin A, 30 min), then with NU-9 (3 μM, 30 min), and finally with Aβ monomer (500 nM, 30 min). Then, AβO puncta bound to dendrites were quantified. Treatment with NU-9 in the absence of lysosome inhibitor mitigated the formation of neuron-binding AβOs (p=2.6 E-5), but treatment with NU-9 in the presence of lysosome inhibitor was unable to do so (p=7.0 E-6) (FIG. 13). Further, treatment with Aβ and lysosome inhibitor in the absence of NU-9 did not lead to the formation of significantly more neuron-binding AβOs compared to treatment with Aβ monomer alone, indicating that lysosome inhibition does not increase AβOs, but only acts to prevent the effect of compound NU-9.

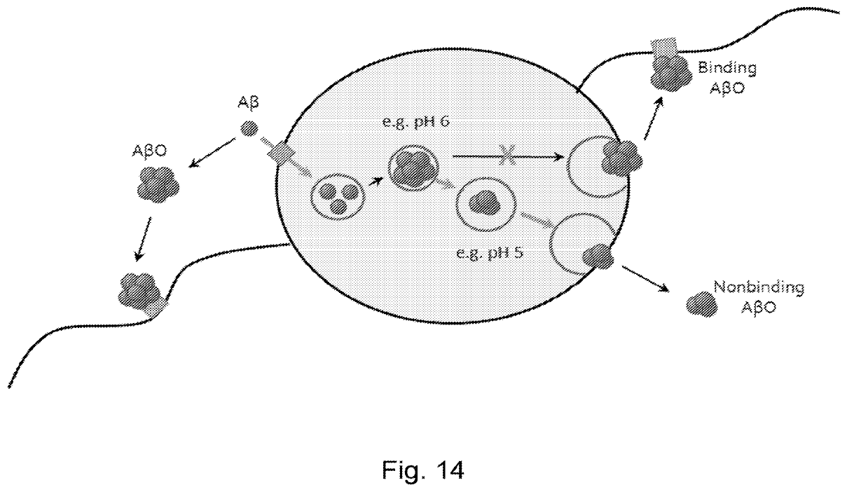

This result suggests that NU-9 acts on a mechanism by which the lysosome promotes the formation of non-binding AβOs at the expense of forming neuron binding AβOs. This could be due to enhanced acidification of the lysosome. Paredes-Rosan et al. showed that, at lower pH (<5), lower molecular weight AβOs are more stable than higher molecular weight AβOs³. Generally, higher molecular weight oligomers are found to bind neurons potently, while lower molecular weight oligomers are unable to do so⁴. Therefore, lysosome acidification could be a reasonable mechanism for the effect of NU-9 on the formation of neuron binding AβOs (FIG. 14).

In the future, Lysotracker or fluorescent lysosomal substrate will be used to measure potential alterations in lysosome activity induced by NU-9. To address the effects of NU-9 on synaptic spine loss induced by AβOs, a previously established synaptic spine assay will be optimized by testing responsiveness of cells to AβO-induced toxicity by co-staining for markers of cell vulnerability, or potentially using other synapse markers such as drebrin antibodies. The spine assay will then be used to determine the effects of NU-9 on AβO-induced toxicity.

### Sources Cited

Pitt, J.; Wilcox, K. C.; Tortelli, V.; Diniz, L. P.; Oliveira, M. S.; Dobbins, C.; Yu, X. W.; Nandamuri, S.; Gomes, F. C. A.; DiNunno, N.; Viola, K. L.; De Felice, F. G.; Ferreira, S. T.; Klein, W. L., Neuroprotective astrocyte-derived insulin/insulin-like growth factor 1 stimulates endocytic processing and extracellular release of neuron-bound Aβ oligomers. Mol Biol Cell 2017, 28 (20), 2623-2636.
Sakono, M.; Zako, T., Amyloid oligomers: formation and toxicity of Aβ oligomers. FEBS J. 2010, 277 (6), 1348-1358.
Paredes-Rosan, C. A.; Valencia, D. E.; Barazorda-Ccahuana, H. L.; Aguilar-Pineda, J. A.; Gómez, B., Amyloid beta oligomers: how pH influences over trimer and pentamer structures? Journal of Molecular Modeling 2019, 26 (1), 1.
Lacor, P. N.; Buniel, M. C.; Chang, L.; Fernandez, S. J.; Gong, Y.; Viola, K. L.; Lambert, M. P.; Velasco, P. T.; Bigio, E. H.; Finch, C. E.; Krafft, G. A.; Klein, W. L., Synaptic targeting by Alzheimer's-related amyloid beta oligomers. J. Neurosci. 2004, 24 (45), 10191-10200.

### Example 3—Characterizing a Lysosome-Dependent Mechanism by which Nu-9 Prevents Neuron-Binding ABO Formation

In this further example, our focus was to characterize the lysosomal mechanism by which addition of NU-9 (3 μM, 30 min) to neurons prior to Aβ monomer (500 nM, 30 min or 24 h) caused an 55-80% reduction in AβOs at synapses compared to treatment with Aβ monomer alone (p=0.001-0.0005 over 3 experiments). Experiments showing: first, that this effect was maintained even when NU-9 was washed out prior to addition of Aβ monomer, second, that in media without cells, NU-9 did not reduce the number of AβOs formed from Aβ monomer (p=0.4), and third, that conditioned media produced by cells treated with NU-9 also did not reduce the number of AβOs formed from Aβ monomer in the absence of cells (p=0.06), support the conclusion that the mechanism of this effect is either intracellular or located at the cell membrane.

Further, the observations that NU-9 does not reduce total Aβ species observable in neuronal cultures (p=0.4), and that treatment of neurons with NU-9 prior to application of Aβ monomer, did not reduce the number of unbound, extracellular AβOs, but in fact increased unbound AβOs twofold (p=0.003), suggested that NU-9 does not act by promoting degradation of Aβ monomer or oligomer, but instead by altering the formation of neuron binding AβO species or by removing AβO binding targets from the cell surface. Finally, we observed that the effect of NU-9 requires lysosome acidity and was abolished in the presence of a lysosome acidification inhibitor (Bafilomycin A, p=7.0 E-6). All these results support the hypothesis that that NU-9 suppresses the formation of a neuron-binding AβO species via a lysosome-dependent mechanism. This mechanism could be directly due to low pH, at which more toxic, high molecular weight AβOs are less stable¹⁻³. Alternately, it could be due to activation of lysosomal cathepsins by NU-9, which also influence Aβ aggregation and are most active when lysosomal pH is low⁴⁻⁷.

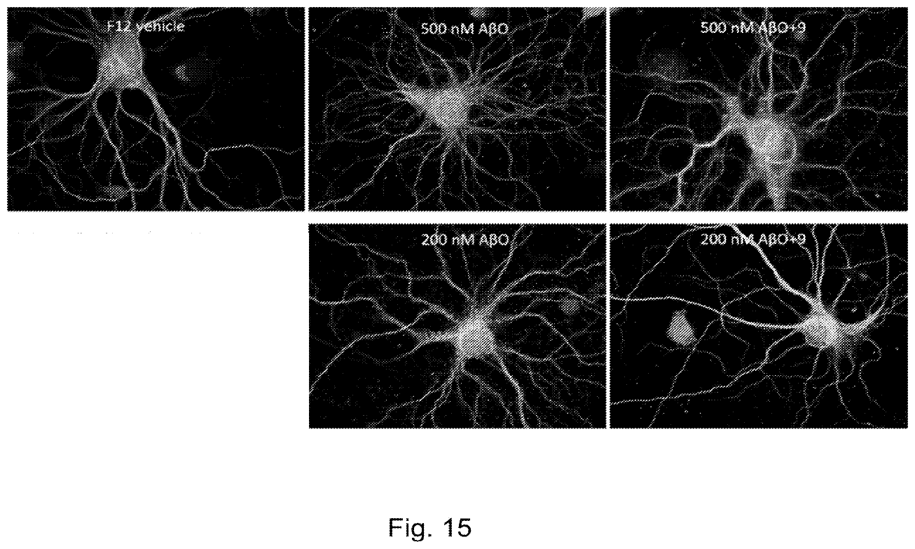

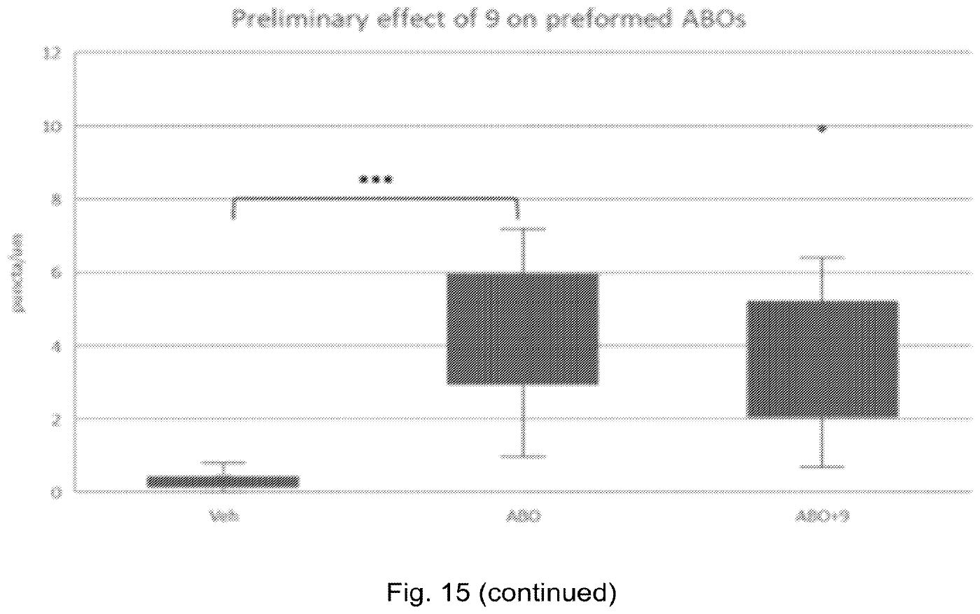

We first verified that NU-9 has no effect on the binding of preformed AβOs to neurons. If NU-9 did have some effect on preformed AβOs, this would support the alternate hypothesis that NU-9 removes AβO binding targets such as NKAα3 or PRP^{c} from the neuronal membrane⁸⁻¹⁰. In these experiments, visual and preliminary qualitative analysis confirmed that NU-9 (3 μM, 30 min, 30 min) has no effect on the binding of preformed AβOs (200 and 500 nM, 30 min, FIG. 15). These experiments further support the conclusion that NU-9 does not merely prevent the binding of pre-formed, binding-capable AβOs, but instead suppresses the formation of a particular species of neuron-binding AβOs.

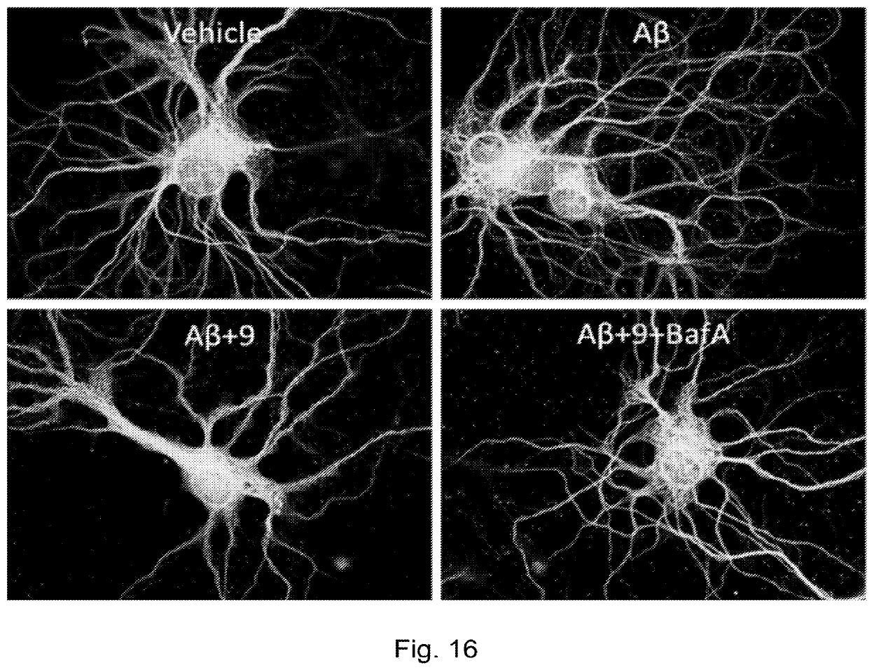

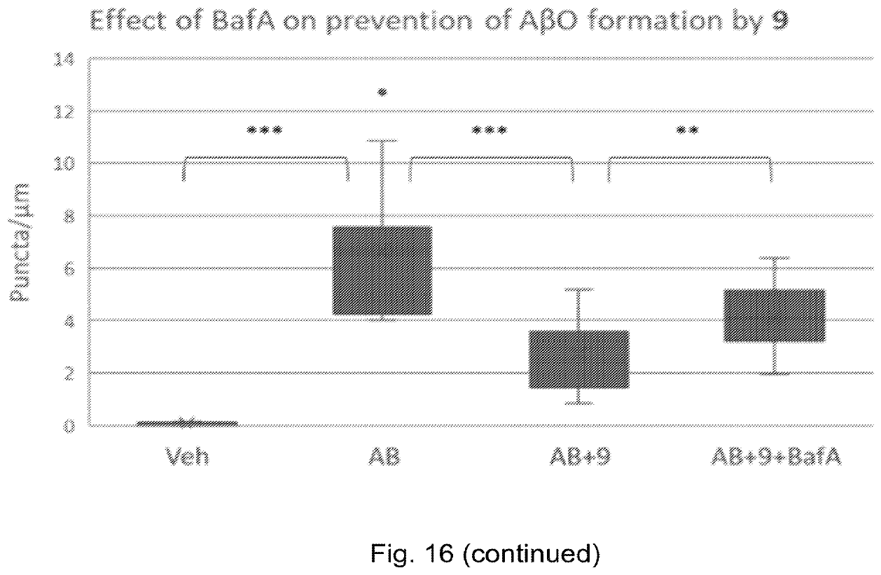

We also verified an experimental result showing that the effect of NU-9 is dependent on lysosome acidity. We observed a replication of this result in the current experiment (FIG. 16). As before, NU-9 (3 μM, 30 min pre-treatment) significantly reduced AβOs bound to neurons treated with Aβ monomer (500 nM, 30 min), and the presence of a lysosome acidification inhibitor (Bafilomyicin A, 100 nM, 30 min prior to NU-9) prevented the effect of NU-9 (p 2.55 E-5, 0.008). This result emphasizes the importance of lysosome acidity for the function of NU-9.

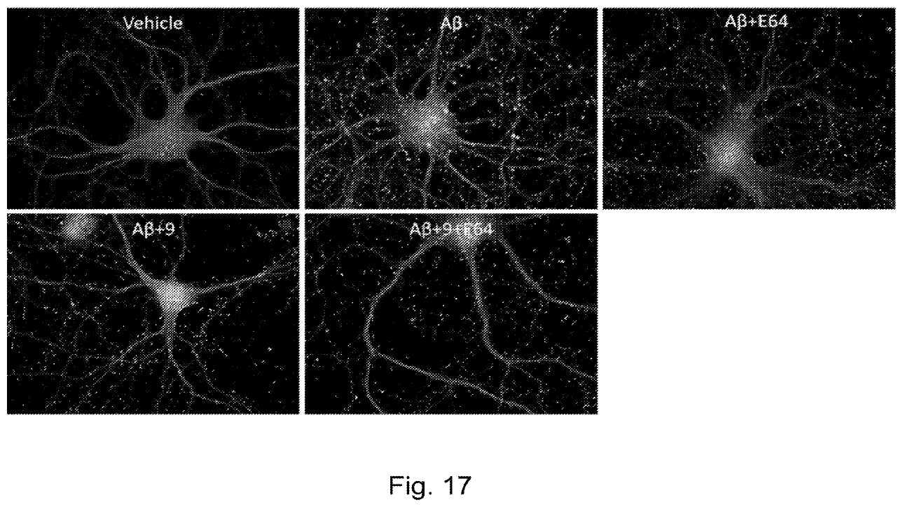

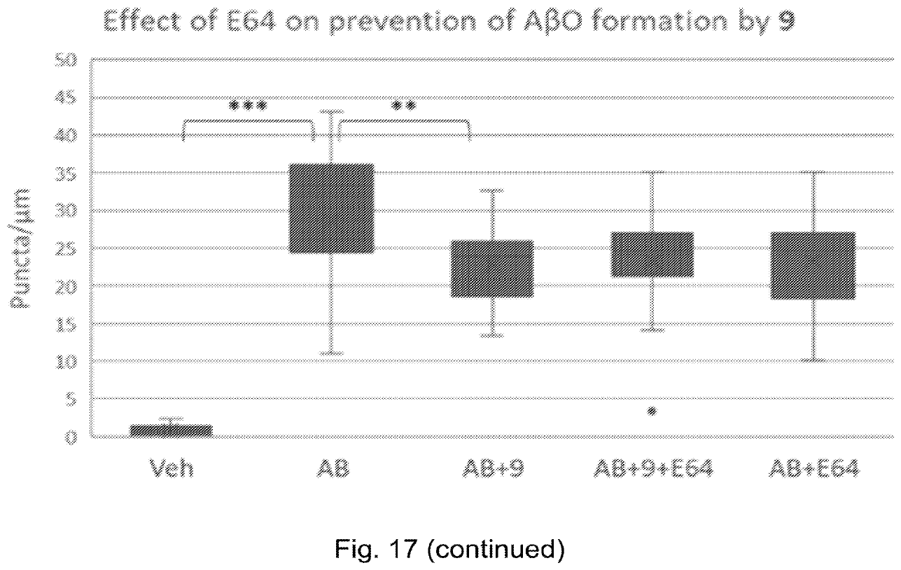

To test whether the lysosome-dependent effect of NU-9 is reliant on the function of the key lysosomal enzymes, cysteine cathepsins, a cysteine cathepsin inhibitor was used. First, mature hippocampal neurons were treated with the cysteine cathepsin inhibitor E64 (10 μM, 24 h), then NU-9 (3 μM, 30 min), and finally Aβ monomer (500 nM, 30 min). As expected, NU-9 significantly reduced the formation of neuron binding AβOs (p 0.005). E64 did not significantly change the effectiveness of NU-9 (p 0.8, FIG. 17). Unusually, E64 alone also significantly reduced the number of bound AβOs (p 0.01). The effects of E64 and NU-9 were not additive, suggesting that they act within the same mechanism. However, the reliance of NU-9 on lysosome acidity suggests that it is not also a cysteine cathepsin inhibitor. Based on this result, it is possible that NU-9 is an activator of a specific cysteine cathepsin, and acts by a different mechanism, but on the same class of target, as E64. It is also possible that NU-9 merely activates lysosome acidification, or endosomal uptake of Aβ for delivery to lysosomes.

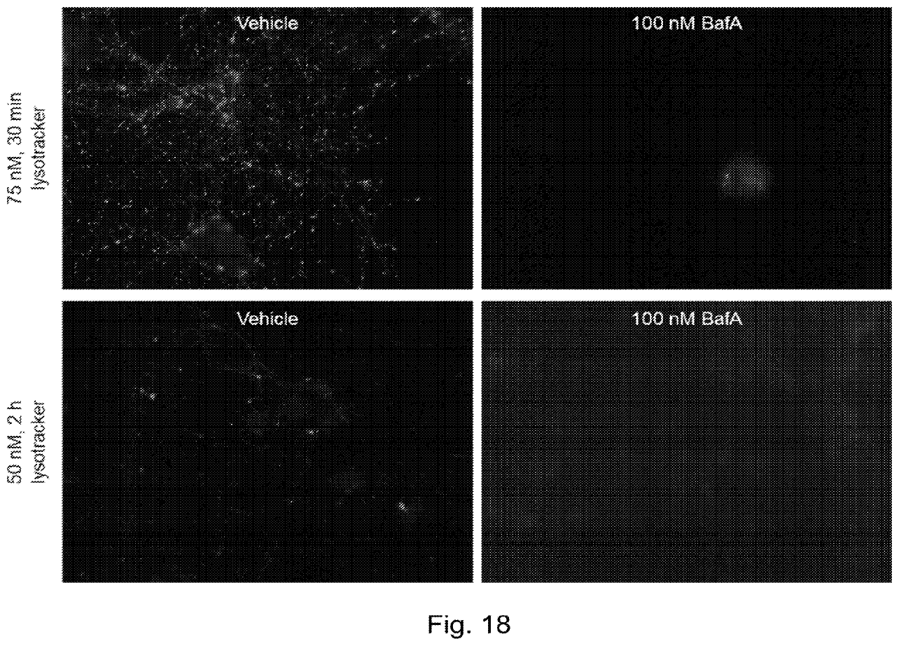

To determine whether NU-9 enhances endo-lysosomal acidity, we also began optimization of a lysosome activity assay using Lysotracker. Lysotracker is a fluorescent dye that is preferentially taken up into acidic compartments and fluoresces more intensely with increasing acidity, and is commonly used in assays of lysosome activation¹¹⁻¹⁴. We found that clear lysosome activity-dependent signal could be observed in mature neurons using a 75 nM, 30 min incubation with lysotracker (FIG. 18). Less intense lysosome activity-dependent signal was also observed using 50 nM, 2 h incubation with Lysotracker. Fixing the cells prior to imaging led to a severe loss of signal. To finalize lysotracker optimization, the lysosome activator Torkinib will be used, to determine whether lysosome activation can be observed.

Another area of investigation this quarter was the effect of NU-9 on the formation of different sizes of AβOs. Evidence from animal models and human samples indicate that that some species of AβOs are more toxic than others, and specifically that higher molecular weight oligomers (>50 kDa) may include the most Aβ-relevant species¹⁵⁻¹⁶. To investigate whether NU-9 reduced the formation of high-molecular weight AβOs, I carried out several experiments using molecular weight cutoff filters of 50 and 100 kDa. However, conflicting results were obtained from different blots. In one experiment, reduction in 50-100 kDa species was observed, alongside an increase in small species (<50 kDa). In another experiment, reduction in most species was observed, except in small species (<50 kDa). The use of sufficient bioreplicates and thorough mixing is important for these experiments. These experiments may be revisited in the future, or the question of the type of AβOs targeted could be addressed through proteomics.

### Sources Cited

Paredes-Rosan, C. A.; Valencia, D. E.; Barazorda-Ccahuana, H. L.; Aguilar-Pineda, J. A.; Gómez, B., Amyloid beta oligomers: how pH influences over trimer and pentamer structures? Journal of Molecular Modeling 2019, 26 (1), 1.
Lacor, P. N.; Buniel, M. C.; Chang, L.; Fernandez, S. J.; Gong, Y.; Viola, K. L.; Lambert, M. P.; Velasco, P. T.; Bigio, E. H.; Finch, C. E.; Krafft, G. A.; Klein, W. L., Synaptic targeting by Alzheimer's-related amyloid beta oligomers. J. Neurosci. 2004, 24 (45), 10191-10200.
Klug, G. M. J. A.; Losic, D.; Supundi; Subasinghe, S.; Aguilar, M.-I.; Martin, L. L.; Small, D. H., β-Amyloid protein oligomers induced by metal ions and acid pH are distinct from those generated by slow spontaneous ageing at neutral pH. European Journal of Biochemistry 2003, 270 (21), 4282-4293.
Bernstein, H.-G.; Keilhoff, G., Putative roles of cathepsin B in Alzheimer's disease pathology: The good, the bad, and the ugly in one? Neural Regen Res 2018, 13 (12), 2100-2101
Turk, B.; Bieth, J. G.; Björk, I.; Dolenc, I.; Turk, D.; Cimerman, N.; Kos, J.; Colic, A.; Stoka, V.; Turk, V., Regulation of the activity of lysosomal cysteine proteinases by pH-induced inactivation and/or endogenous protein inhibitors, cystatins. Biol Chem Hoppe Seyler 1995, 376 (4), 225-30.
Hook, G.; Yu, J.; Toneff, T.; Kindy, M.; Hook, V., Brain pyroglutamate amyloid-β is produced by cathepsin B and is reduced by the cysteine protease inhibitor E64d, representing a potential Alzheimer's disease therapeutic. J Alzheimers Dis 2014, 41 (1), 129-149.
Schilling, S.; Lauber, T.; Schaupp, M.; Manhart, S.; Scheel, E.; Bohm, G.; Demuth, H. U., On the seeding and oligomerization of pGlu-amyloid peptides (in vitro). Biochemistry 2006, 45 (41), 12393-9.
DiChiara, T.; DiNunno, N.; Clark, J.; Bu, R. L.; Cline, E. N.; Rollins, M. G.; Gong, Y.; Brody, D. L.; Sligar, S. G.; Velasco, P. T.; Viola, K. L.; Klein, W. L., Alzheimer's Toxic Amyloid Beta Oligomers: Unwelcome Visitors to the Na/K ATPase alpha3 Docking Station. Yale J Biol Med 2017, 90 (1), 45-61.
Komura, H.; Kakio, S.; Sasahara, T.; Arai, Y.; Takino, N.; Sato, M.; Satomura, K.; Ohnishi, T.; Nabeshima, Y.-i.; Muramatsu, S.-i.; Kii, I.; Hoshi, M., Alzheimer Aβ Assemblies Accumulate in Excitatory Neurons upon Proteasome Inhibition and Kill Nearby NAKα3 Neurons by Secretion. iScience 2019, 13, 452-477.
Laurén, J.; Gimbel, D. A.; Nygaard, H. B.; Gilbert, J. W.; Strittmatter, S. M., Cellular prion protein mediates impairment of synaptic plasticity by amyloid-beta oligomers. Nature 2009, 457 (7233), 1128-32.
Leeman, D. S.; Hebestreit, K.; Ruetz, T.; Webb, A. E.; McKay, A.; Pollina, E. A.; Dulken, B. W.; Zhao, X.; Yeo, R. W.; Ho, T. T.; Mahmoudi, S.; Devarajan, K.; Passegué, E.; Rando, T. A.; Frydman, J.; Brunet, A., Lysosome activation clears aggregates and enhances quiescent neural stem cell activation during aging. Science 2018, 359 (6381), 1277.
Zhou, J.; Tan, S. H.; Nicolas, V.; Bauvy, C.; Yang, N. D.; Zhang, J.; Xue, Y.; Codogno, P.; Shen, H. M., Activation of lysosomal function in the course of autophagy via mTORC1 suppression and autophagosome-lysosome fusion. Cell Res 2013, 23 (4), 508-23.
Zhang, J.; Wang, J.; Wong, Y. K.; Sun, X.; Chen, Y.; Wang, L.; Yang, L.; Lu, L.; Shen, H.-M.; Huang, D., Docetaxel enhances lysosomal function through TFEB activation. Cell Death Dis 2018, 9 (6), 614-614.
Meng, S. F.; Mao, W. P.; Wang, F.; Liu, X. Q.; Shao, L. L., The relationship between Cd-induced autophagy and lysosomal activation in WRL-68 cells. J Appl Toxicol 2015, 35 (11), 1398-405.
Lesné, S.; Koh, M. T.; Kotilinek, L.; Kayed, R.; Glabe, C. G.; Yang, A.; Gallagher, M.; Ashe, K. H., A specific amyloid-beta protein assembly in the brain impairs memory. Nature 2006, 440 (7082), 352-7.
Upadhaya, A. R.; Lungrin, I.; Yamaguchi, H.; Fandrich, M.; Thal, D. R., High-molecular weight Aβ oligomers and protofibrils are the predominant Aβ species in the native soluble protein fraction of the AD brain. J Cell Mol Med 2012, 16 (2), 287-95.
Fukumoto, H.; Tokuda, T.; Kasai, T.; Ishigami, N.; Hidaka, H.; Kondo, M.; Allsop, D.; Nakagawa, M., High-molecular-weight beta-amyloid oligomers are elevated in cerebrospinal fluid of Alzheimer patients. Faseb J 2010, 24 (8), 2716-26.

### Example 4—NU-9 Acts Via a Lysosome-Dependent Mechanism

In this example, the inventors further investigate the mechanism of action of NU-9 for preventing AβO formation and accumulation on neurons.

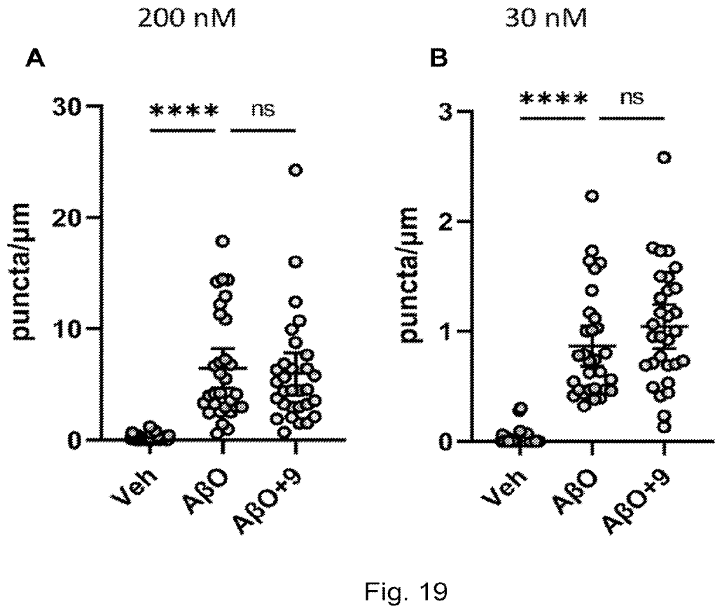

NU-9 had no effect on the binding of the AβOs to hippocampal neurons. This result supports the conclusion that NU-9 does not prevent binding of preformed AβOs but instead interferes with a distinct cellular mechanism of AβO formation and accumulation (FIG. 19).

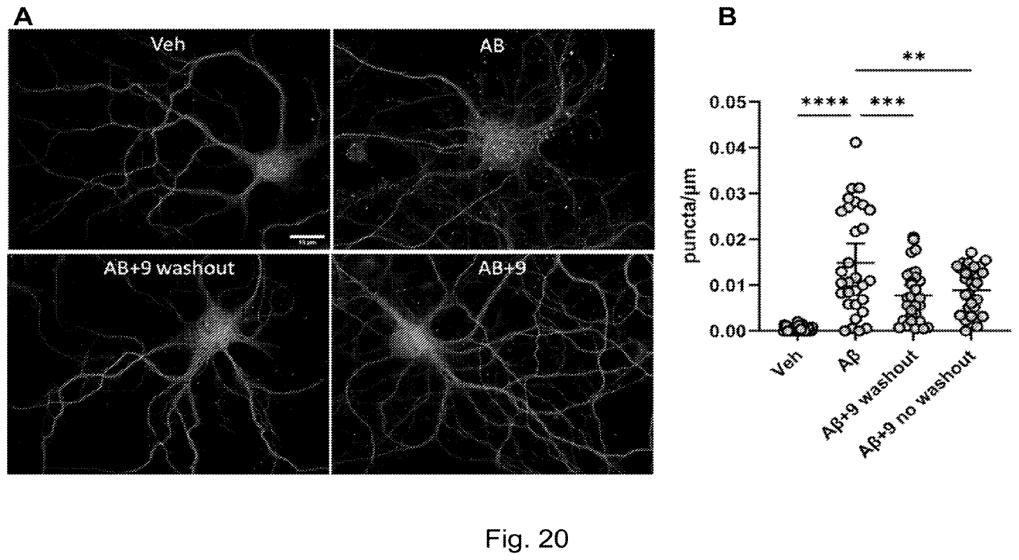

In this experiment, it was observed that NU-9 reduced the number of AβOs bound to dendrites, even when NU-9 was washed out prior to application of Aβ monomer. This result supports a cellular mechanism for NU-9 (FIG. 20).

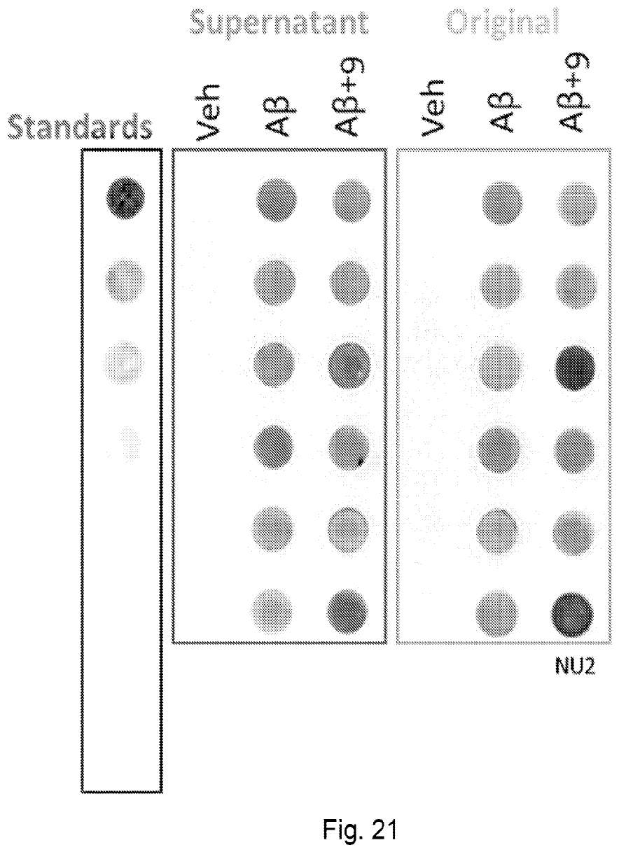

NU-9 did not alter the number of unbound, extracellular AβOs, suggesting a specific effect on accumulation of toxic, cell-binding species (FIG. 21).

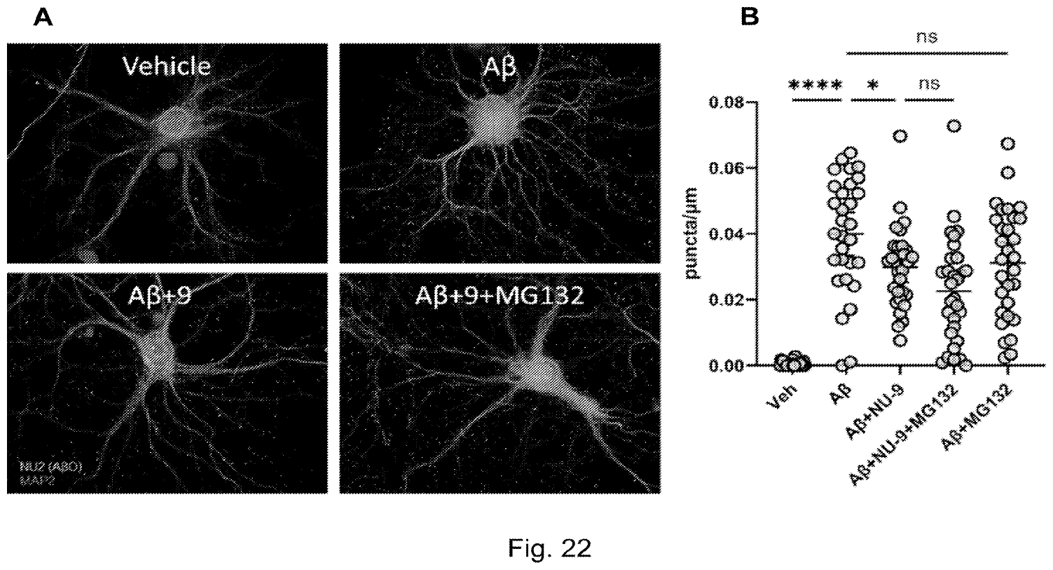

Proteasome inhibition using 100 nM MG132 had no effect on formation of AβOs or reduction of AβO accumulation by NU-9. This suggests that NU-9 may not act in a proteasome-dependent manner (FIG. 22).

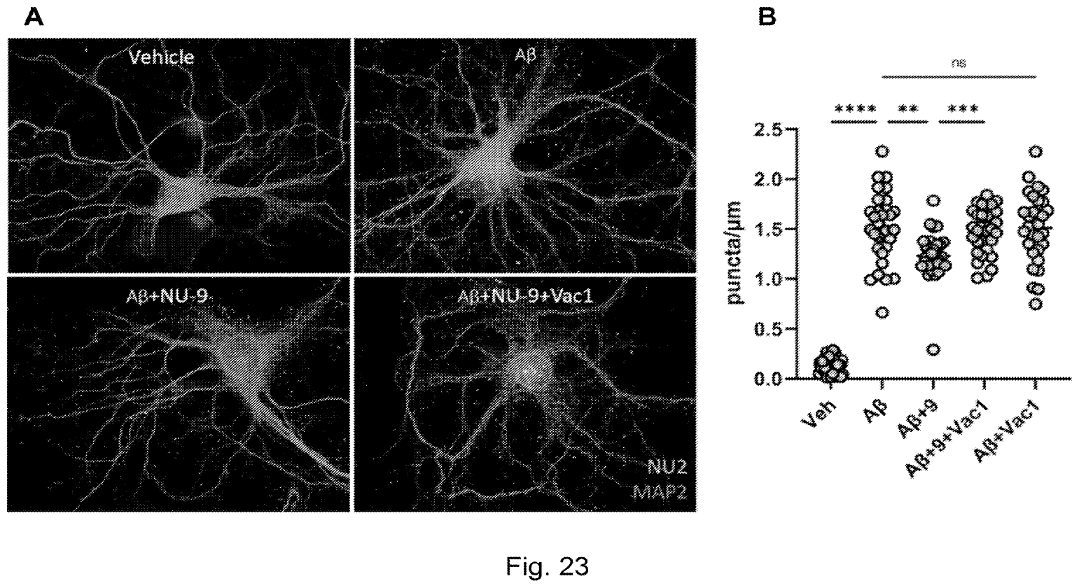

Inhibition of lysosome maturation and exocytosis by vacuolin-1 prevented the effectiveness of NU-9. This supports the conclusion that NU-9 acts via a lysosome-dependent mechanism (FIG. 23).

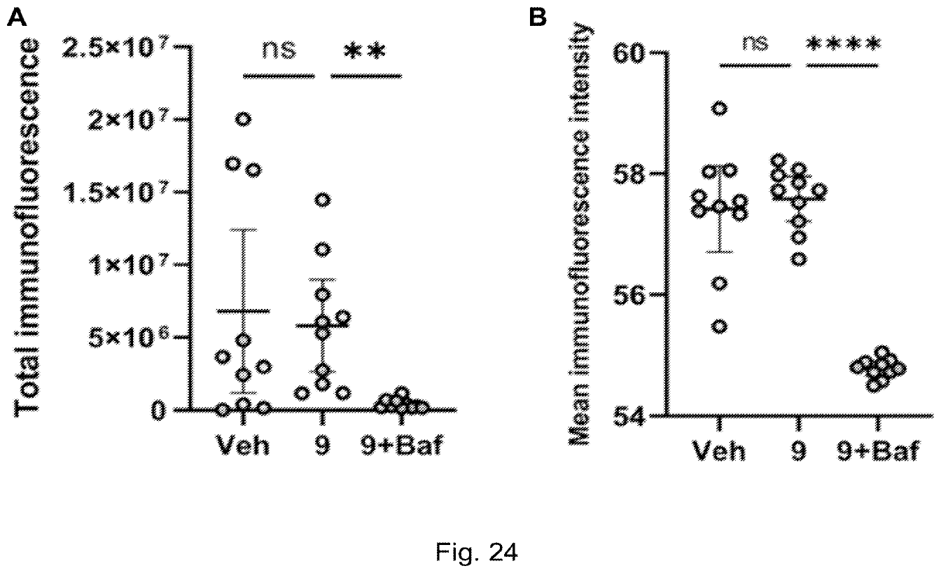

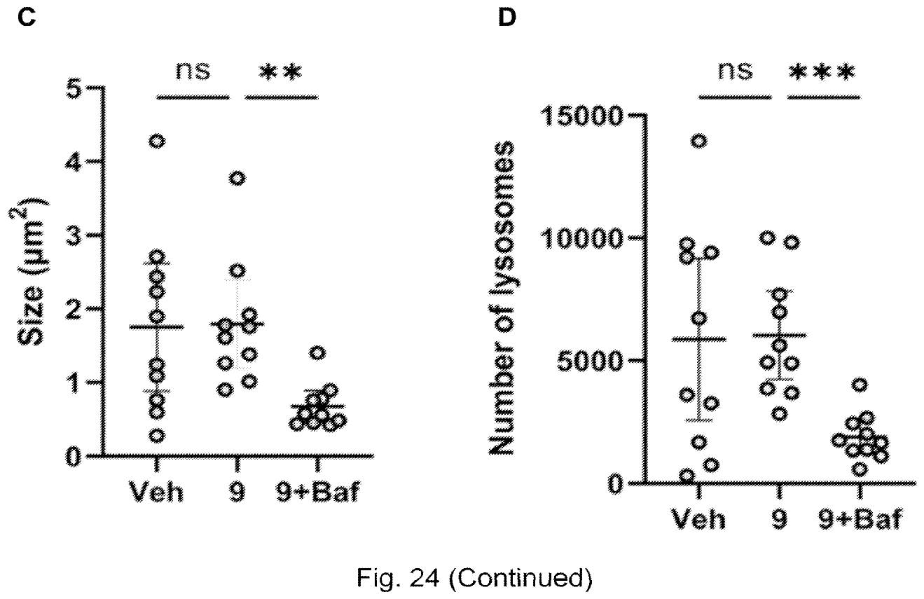

Following 30-minute treatment of NU-9, integrated fluorescent density of lysotracker, indicating acidic lysosomes, was not altered, although it was significantly reduced with the addition of the control Bafilomycin A. Lysosome number, size, and acidity were unchanged with NU-9. These results support the conclusion that NU-9 does not act by altering lysosome number or acidity (FIG. 24).

Addition of 10 μM CA-074, a cathepsin B inhibitor, to neurons for 24 h prior to the addition of NU-9 prevented the effect of NU-9. This supports the conclusion that the effect of NU-9 to reduce AβO accumulation on neurons is cathepsin B dependent (FIG. 25).

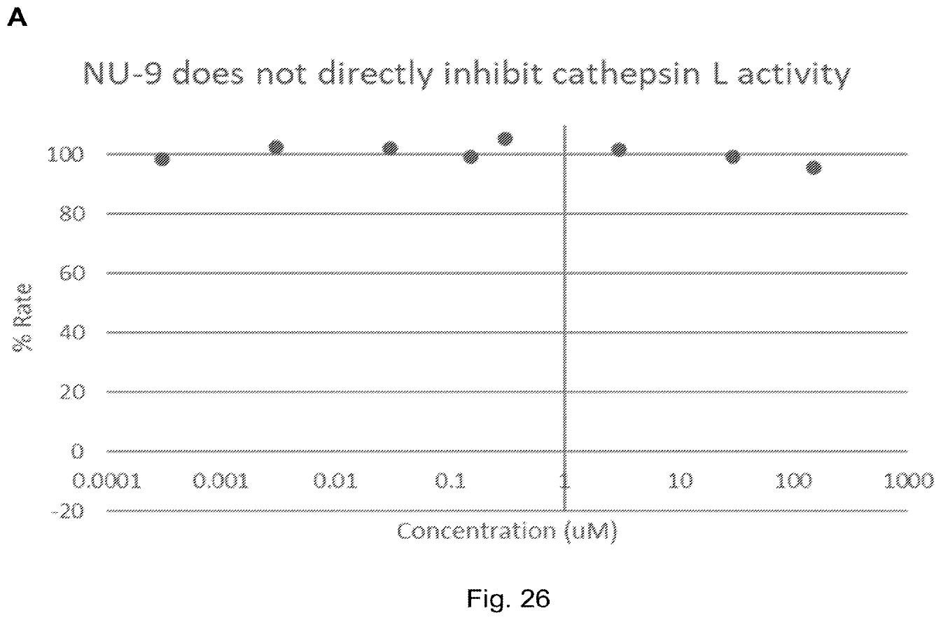

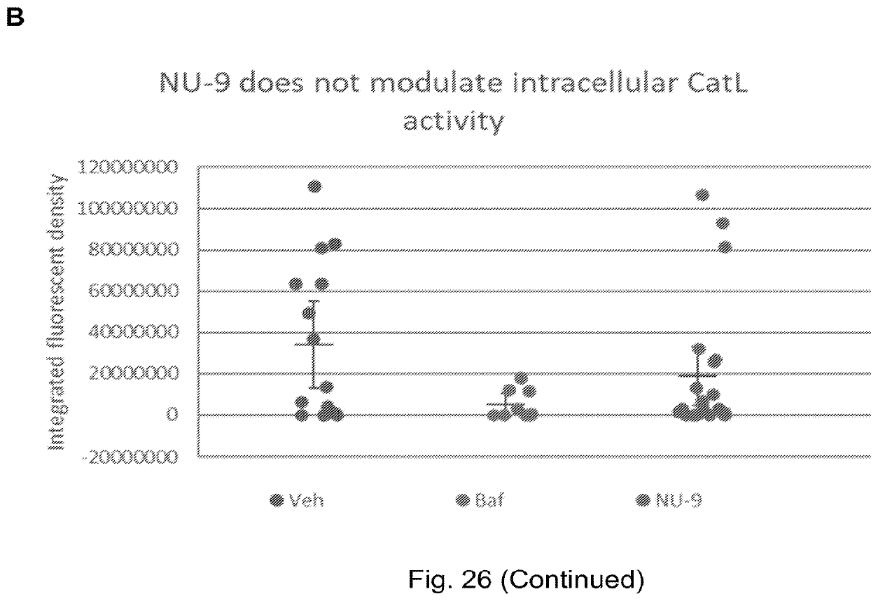

No effect of 0.0003-150 μM NU-9 was observed on the cathepsin L activity as measured by the production rate of 7-AMC from Z-FR-AMC by purified cathepsin L enzyme. In this assay, the standard inhibitor Z-FY-CHO reduced cathepsin L activity over the same concentration range. This result supports the conclusion that NU-9 does not directly inhibit cathepsin L. NU-9 was also not observed to modulate intracellular cathepsin L activity measured in hippocampal neuron cultures (FIG. 26).

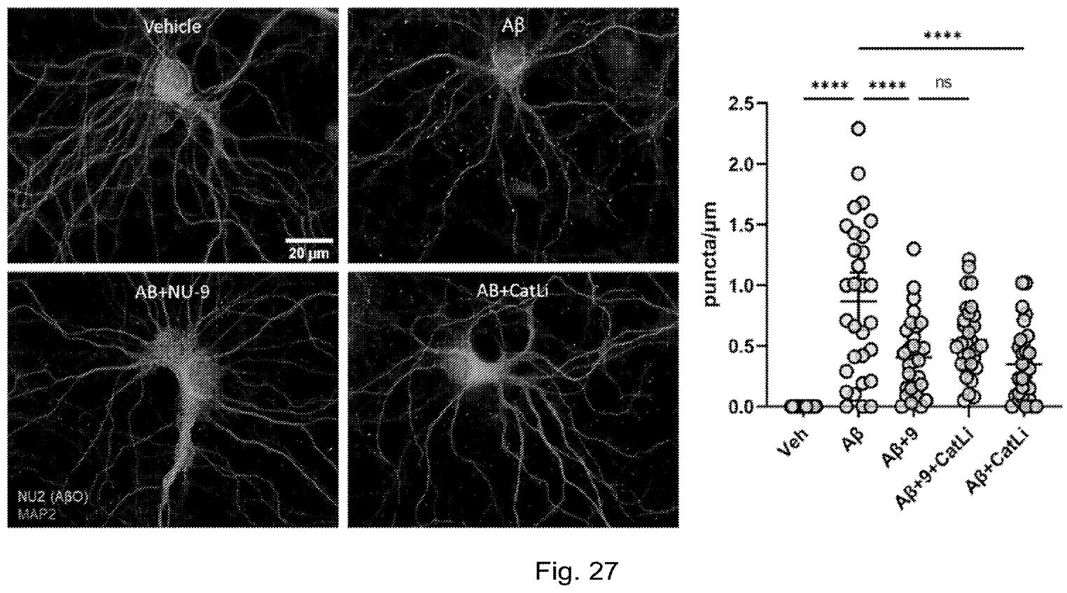

Inhibition of cathepsin L prevents AβO accumulation, mimicking the effect of NU-9. Neurons were pre-treated first for 1 h with 10 μM of Cathepsin L inhibitor Z-FY-CHO (aka SB-412515, 10 μM), then for 30 minutes with 3 μM NU-9, and finally for 30 minutes to 500 nM Aβ. The cathepsin L inhibitor treatment mimicked the effect of NU-9 (p<0.0001) (FIG. 27). This trend was replicated in a follow-up experiment with a different cell culture.

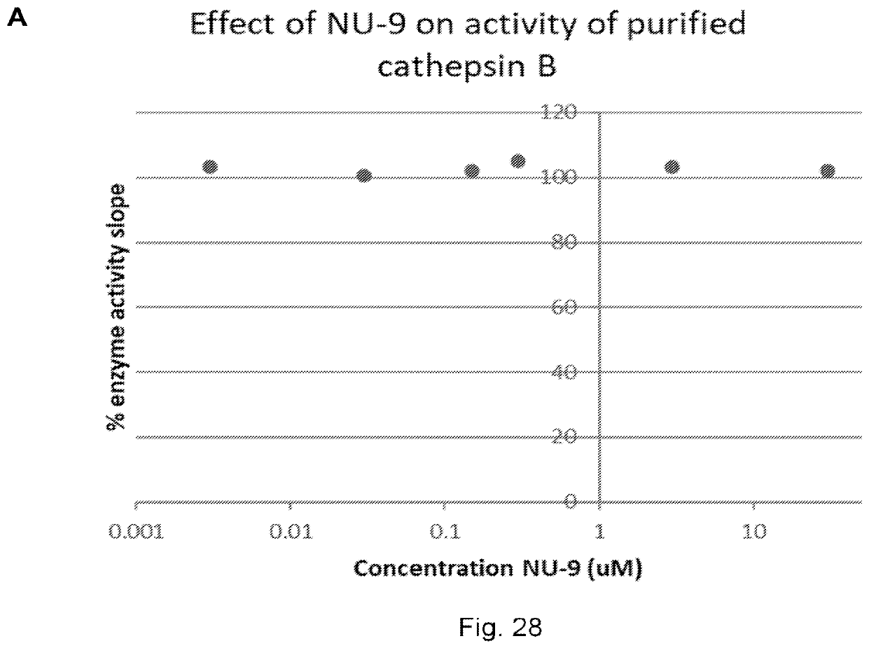

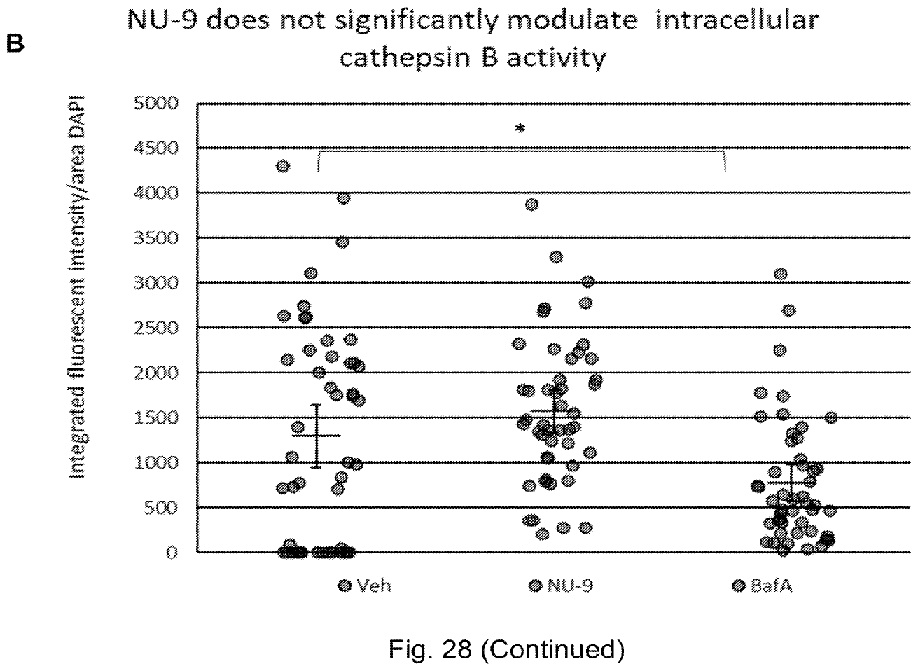

Minimal effect of NU-9 (0.0003-30 μM) was observed on activity (cleavage of Z-RR-AMC to 7-AMC) of purified cathepsin B enzyme. Clear inhibition of enzyme activity was observed using over the same concentration range of the standard cathepsin B inhibitor CA-074. This result supports the conclusion that NU-9 may not directly activate cathepsin B. In a cell-based assay, NU-9 also was not observed to alter cathepsin B activity in hippocampal neurons (FIG. 28).

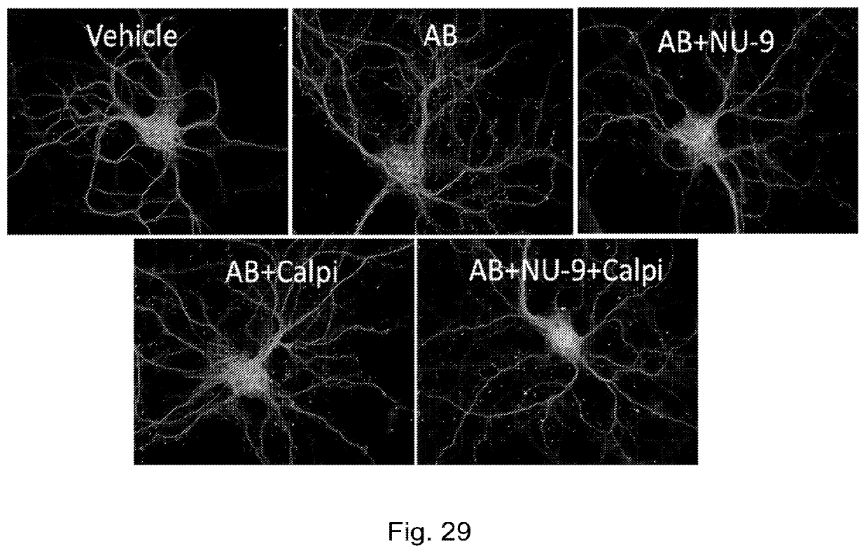

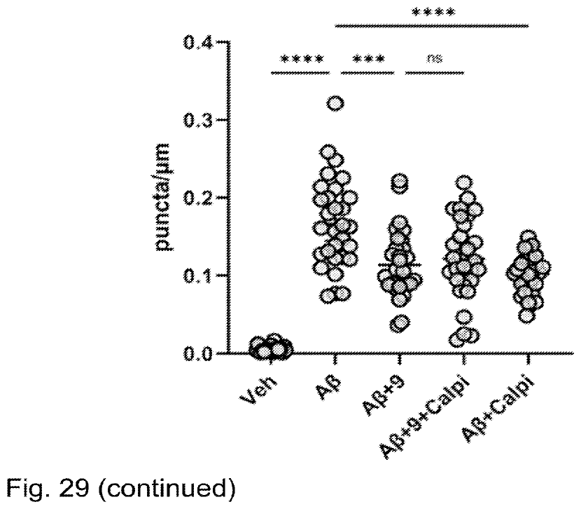

Calpain inhibition may mimic the effect of NU-9. To test the effect of calpain inhibition on the effectiveness of NU-9 and AβO accumulation, we used the specific inhibitor MDL-28170. We applied 10 μM MDL-28170 to mature hippocampal neurons for 30 min, then added 3 μM NU-9, and finally introduced 500 nM Aβ42. Inhibition of calpain was observed to mimic the effect of NU-9, also reducing AβO accumulation (p<0.0001) (FIG. 29). The reduction in AβO accumulation by both calpain and cathepsin L supports the conclusion that AβO accumulation is dependent upon the activity of these intracellular cysteine cathepsins.

The present example also demonstrates the ability to detect candidate compounds that modulates amyloid-beta oligomerization by comparison to NU-9.

In conclusion, the inventors discovered that NU-9 does not reduce AβO toxicity by interfering with AβO binding to neurons, rather, NU-9 acts via a cellular, lysosome-dependent mechanism to prevent AβO toxicity.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of treating Alzheimer's disease in a subject in need thereof comprising: administering an effective amount of (S)-5-(1-(3,5-bis(trifluoromethyl)phenoxy)ethyl)cyclohexane-1,3-dione, or a suitable pharmaceutical salt thereof, to the subject to treat Alzheimer's disease in the subject.

2. The method of claim 1, wherein the method treats memory loss in the subject.

3. The method of claim 1 or 2, wherein the subject suffers from amyloid-beta oligomerization.

4. A method of treating or preventing a disease or disorder associated with amyloid-beta oligomerization in a subject in need thereof, the method comprising administering an effective amount of (S)-5-(1-(3,5-bis(trifluoromethyl)phenoxy)ethyl)cyclohexane-1,3-dione, or a suitable pharmaceutical salt thereof, to the subject to treat a disease or disorder associated with amyloid-beta oligomerization in the subject.

5. The method of claim 4, wherein the disease or disorder is selected from Alzheimer's disease, cerebral amyloid angiopathy (CAA), inflammatory cerebral amyloid angiopathy, and cerebral amyloidoma.

6. The method of any one of claims 4-5, wherein the method treats memory loss in the subject.

7. A method of modulating amyloid beta oligomerization activity in a subject's brain comprising administering an effective amount of (S)-5-(1-(3,5-bis(trifluoromethyl)phenoxy)ethyl)cyclohexane-1,3-dione, or a suitable pharmaceutical salt thereof, to the subject to modulate amyloid-beta oligomerization activity in the subject's brain.

8. The method of claim 7, wherein the method inhibits formation of neuron binding amyloid beta oligomers.

9. The method of any one of claims 7-8, wherein the method promotes formation of non-binding amyloid beta oligomers.

10. The method of any one of claims 7-9, wherein the subject suffers from Alzheimer's disease.

11. The method of any one of claims 7-10, wherein the method treats memory loss in the subject.

12. The method of any one of claims 7-11, wherein the subject suffers from amyloid-beta oligomerization in the brain.

13. The method of any one of claims 1-12, wherein the subject is administered a daily dose of the compound of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values.

14. The method of any one of claims 1-13, wherein the compound is administered orally.

15. The method of any one of claims 1-14 further comprising administering a cholinesterase inhibitor and/or a N-methyl-D-aspartate receptor antagonist.

16. The method of claim 15, wherein the method comprises administering the cholinesterase inhibitor and the cholinesterase inhibitor is selected from galantamine, rivastigmine, and donepezil.

17. The method of claim 15, wherein the method comprises administering the N-methyl-D-aspartate receptor antagonist and the N-methyl-D-aspartate receptor antagonist comprises memantine.

18. A method for detecting candidate compounds that modulates amyloid-beta oligomerization in the presence of cells comprising:
(i) culturing cells with amyloid-beta peptide in the presence and absence of a candidate compound and contacting control cells with amyloid beta peptide in the presence and absence of a control compound;
(ii) detecting one or more parameters related to oligomerization of amyloid-beta in the cells of step (i);
(iii) generating a test index by calculating a change in the one or more parameters between the cells cultured in the presence and absence of the candidate compound and generating a control index by calculating a change in the one or more parameters between the cells cultured in the presence and absence of the control compound;
wherein the control compound is (S)-5-(1-(3,5-bis(trifluoromethyl)phenoxy)ethyl)cyclohexane-1,3-dione, or a pharmaceutically acceptable salt thereof, and
wherein, if the value of the test index is equal to, or improved, as compared to the value of the control index, then the candidate compound modulates amyloid-beta oligomerization.

19. The method of claim 18, wherein the cells are neurons or are derived from neurons.

20. The method of claim 19, wherein the cells are E18 hippocampal neurons.

21. The method of any one of claims 18-20, wherein the one or more parameters comprise detecting amyloid-beta oligomers (AβO) bound to the cells.

22. A unit dosage package comprising:
(i) (S)-5-(1-(3,5-bis(trifluoromethyl)phenoxy)ethyl)cyclohexane-1,3-dione, or a pharmaceutically acceptable salt thereof; and
(ii) a cholinesterase inhibitor or an N-methyl-D-aspartate receptor antagonist.

23. The unit dosage package of claim 22, wherein the cholinesterase inhibitor is selected from galantamine, rivastigmine, and donepezil.

24. The unit dosage package of claim 22, wherein the N-methyl-D-aspartate receptor antagonist comprises memantine.

25. A pharmaceutical composition comprising:
(i) (S)-5-(1-(3,5-bis(trifluoromethyl)phenoxy)ethyl)cyclohexane-1,3-dione, or a pharmaceutically acceptable salt thereof;
(ii) a cholinesterase inhibitor or N-methyl-D-aspartate receptor antagonist; and
(iii) a pharmaceutically acceptable carrier or excipient.

26. The pharmaceutical composition of claim 25, wherein the cholinesterase inhibitor is selected from galantamine, rivastigmine, and donepezil.

27. The pharmaceutical composition of claim 25, wherein the N-methyl-D-aspartate receptor antagonist comprises memantine.


SEQUENCE LISTING (4 sequences)

SEQ ID NO: 1 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 40 aa, Homo sapiens
DAEFRHDSGYEVHHQKLVFFAEDVGSNKGAIIGLMVGGVV

SEQ ID NO: 3 - protein, 42 aa, Homo sapiens [sequence omitted]

SEQ ID NO: 4 - protein, 40 aa, Homo sapiens
  SITE 1: note = MISC_FEATURE - Pyroglutamate
EFRHDSGYEVHHQKLVFFAEDVGSNKGAIIGLMVGGVVIA